SEQUENCE LISTING (8 sequences)

SEQ ID NO: 1 - protein, 227 aa, synthetic construct
DKTHTCPPCPVPELLAGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHNHYTQKSLSLSPGK

SEQ ID NO: 2 - DNA, 681 bp, synthetic construct
  source 1..681: mol_type = other DNA
gacaaaactcacacatgcccaccgtgcccagtacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccgtgactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctctcgtgctggactccgacggctccttcttcctctacagcaagctcaccgtggacaagagcaggtggcagcaggggaacgtcttctcatgctccgtgctgcatgaggctctgcataaccactacacgcagaagagcctctccctgtccccgggtaaa

SEQ ID NO: 3 - protein, 227 aa, synthetic construct
DKTHTCPPCPAPELLAGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSLDELTKNQVSLTCLVKGFYPSDIAVEWESNGQPENNYKTTPLVLDSDGSFFLYSKLTVDKSRWQQGNVFSCSVLHEALHNHYTQKSLSLSPGK

SEQ ID NO: 4 - DNA, 681 bp, synthetic construct
  source 1..681: mol_type = other DNA
gacaaaactcacacatgcccaccgtgcccagcacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccgtgactggctgaatggcaaggagtacaagtgcaaggtctccaacaaagccctcccagcccccatcgagaaaaccatctccaaagccaaagggcagccccgagaaccacaggtgtacaccctgcccccatccctggatgagctgaccaagaaccaggtcagcctgacctgcctggtcaaaggcttctatcccagcgacatcgccgtggagtgggagagcaatgggcagccggagaacaactacaagaccacacctctcgtgctggactccgacggctccttcttcctctacagcaagctcaccgtggacaagagcaggtggcagcaggggaacgtcttctcatgctccgtgctgcatgaggctctgcataaccactacacgcagaagagcctctccctgtccccgggtaaa

SEQ ID NO: 5 - protein, 227 aa, synthetic construct
DKTHTCPPCPVPELLAGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSLDELTKNQVSLTCLVKGFYPSDIAVEWESNGQPENNYKTTPLVLDSDGSFFLYSKLTVDKSRWQQGNVFSCSVLHEALHNHYTQKSLSLSPGK

SEQ ID NO: 6 - DNA, 681 bp, synthetic construct
  source 1..681: mol_type = other DNA
gacaaaactcacacatgcccaccgtgcccagtacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccgtgactggctgaatggcaaggagtacaagtgcaaggtctccaacaaagccctcccagcccccatcgagaaaaccatctccaaagccaaagggcagccccgagaaccacaggtgtacaccctgcccccatccctggatgagctgaccaagaaccaggtcagcctgacctgcctggtcaaaggcttctatcccagcgacatcgccgtggagtgggagagcaatgggcagccggagaacaactacaagaccacacctctcgtgctggactccgacggctccttcttcctctacagcaagctcaccgtggacaagagcaggtggcagcaggggaacgtcttctcatgctccgtgctgcatgaggctctgcataaccactacacgcagaagagcctctccctgtccccgggtaaa

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 681 bp, Homo sapiens
  source 1..681: mol_type = genomic DNA
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

(19) United States
(12) Patent Application Publication
(10) Pub. No.: US 2025/0297031 A1
(43) Pub. Date: Sep. 25, 2025

(54) HUMAN FC VARIANTS HAVING IMPROVED FCGRIIA BINDING SELECTIVITY

(71) Applicant: KOREA UNIVERSITY RESEARCH AND BUSINESS FOUNDATION, Seoul (KR)

(72) Inventors: Sang Taek JUNG, Seoul (KR); Woo Hyung KO, Seoul (KR); Sanghwan KO, Seoul (KR); Migyeong JO, Seoul (KR); Munsu KYUNG, Gimpo-si (KR); Suyeon KIM, Seoul (KR)

(73) Assignee: KOREA UNIVERSITY RESEARCH AND BUSINESS FOUNDATION, Seoul (KR)

(21) Appl. No.: 18/864,138

(22) PCT Filed: Jun. 16, 2023
(86) PCT No.: PCT/KR2023/008402

§ 371 Date: Nov. 8, 2024

(30) Foreign Application Priority Data
Jun. 29, 2022 (KR) 10-2022-0079827

(51) Int. Cl. C07K 16/32; A61K 39/00; A61P 35/00

(52) U.S. Cl. CPC C07K 16/32; A61P 35/00; A61K 2039/505; C07K 2317/14; C07K 2317/52; C07K 2317/72; C07K 2317/732; C07K 2317/92; C07K 2317/94

(57) ABSTRACT

Provided is an Fc variant which has improved half-life by binding to and unbinding from FcRn in a pH-dependent manner, and which has improved selective binding to Fcγ receptors. Compared to a wild-type human antibody Fc domain and conventional antibodies approved as antibody therapeutic agents, the present human antibody Fc domain variants have a lower capacity to bind to immune-inhibiting receptors FcγRIIb and FcγRIIIb and have a higher capacity to bind to immune-activating receptor FcγRIIa (increased A/I ratio), thereby having a remarkably improved effector function and having maximized half-life in blood in which excellent pH-selective FcRn binding and unbinding capacity is exhibited, and thus bind to numerous peptide drug therapeutics having short half-life and retention time in the body so that long-term drug efficacy through increased blood half-life can be exhibited, and can maximize the immune mechanism of therapeutic protein drugs.

### TECHNICAL FIELD

The present disclosure relates to an Fc variant having an improved capacity to bind to human FcγRIIa, which induces phagocytosis of IgG antibodies against target cells and molecules, and to a technology for improving an effector function of a therapeutic antibody against a target.

### BACKGROUND ART

Protein therapeutic agents have very high specificity for disease targets and low side effects and toxicity and thus are widely used in clinical trials by rapidly replacing non-specific small molecule compound therapeutic agents, and among protein therapeutic agents currently used in clinical trials, antibody therapeutic agents and Fc-fusion protein therapeutic agents that are fused with the Fc domain of an antibody make up the largest part. Therapeutic antibodies show very high specificity for targets compared to conventional small molecule drugs, have not only low biotoxicity and few side effects, but also an excellent blood half-life of about 3 weeks, and thus are considered as one of the most effective cancer therapy methods. In fact, large pharmaceutical companies and research institutes around the world are accelerating the research and development of therapeutic antibodies that specifically bind to cancer cells, including cancer-causing factors, to effectively remove the cancer cells. Companies for developing therapeutic antibody drugs mainly consist of pharmaceutical companies such as Roche, Amgen, Johnson & Johnson, Abbott, and BMS. Particularly, Roche includes representative products of Herceptin, Avastin, Rituxan, and the like for anti-cancer treatment, and these three therapeutic antibodies not only achieved large profits, achieving sales of approximately $19.5 billion in the global market in 2012, but are also leading the antibody drug market of the world. Johnson & Johnson, which developed Remicade, is also growing rapidly in the global antibody market due to increased sales, and pharmaceutical companies such as Abbott and BMS are also known to have many therapeutic antibodies in the final stages of development. As a result, biopharmaceuticals containing therapeutic antibodies that are specific for disease targets and have low side effects are rapidly replaced in the global pharmaceutical market, where small molecule drugs had the initiative. The antibody provides a linkage between the humoral and cellular immune systems, and a Fab region of the antibody recognizes an antigen, whereas an Fc domain region binds to a receptor (Fc receptor or FcR) for an antibody (immunoglobulin) on a cell that is differentially expressed by all immune competent cells and has a different mechanism depending on a type of FcγR expressed on the surface of the binding immune cell. An Fc receptor binding site on the Fc domain of the antibody binds to the Fc receptor (FcR) on the cell, and the antibody binds to the Fc receptor on the cell surface through the Fc domain to trigger a variety of important biological responses, including control of phagocytosis and destruction of antibody-coated particles, removal of immune complexes, lysis of antibody-coated target cells by killing cells (antibody-dependent cell-mediated cytotoxicity, or ADCC), release of inflammatory mediators, placental transfer and immunoglobulin production (Deo, Y. M. et al., Immunol. Today 18 (3): 127-135 (1997)). As such, the Fc domain plays a critical role in the collection of immune cells and ADCC and antibody dependent cell-mediated phagocytosis (ADCP). In particular, the ADCC and ADCP functions, which are the effector functions of the antibody, depend on interaction with Fc receptors present on the surfaces of many cells. Accordingly, attempts to modify antibodies to collect specific cells may be very important in the field of treatment. Human Fc receptors are classified into five types, and four of the five major types of human FcγRs induce immune activating or inflammatory responses, and FcγRIIb induces immune inhibiting or anti-inflammatory responses. Since the type of immune cell to be collected is determined depending on which Fc receptor (e.g., immune-activating receptor: FcγRI, FcγRIIa, FcγRIIIa/immune-inhibiting receptor: FcγRIIb) the antibody binds to, attempts to modify the antibodies so as to collect specific cells are very important in the therapeutic field. Currently, the action of antibodies as pharmaceuticals has a direct method by binding to an antigen to inhibit the action of the antigen, and an indirect antigen removal method by effector cells (natural killer cells, macrophages, etc.) having an Fc gamma region and an Fc gamma receptor (FCGR) of the antibody binding to the antigen. In the case of an antibody therapeutic agent that is being developed recently, the effect of the drug is increased through an ADCC mechanism in which the effector cell recognizes the Fc gamma region of the antibody to attack and remove the antigen while preventing the action of the antigen due to binding to the antigen. In particular, in the treatment of viral infectious diseases, mechanisms of feeding virus particles and infected cells and antigen presenting through ADCP of antibodies are very important. Unlike other immune cells (e.g., T-cells, B-cells, NK cells), the macrophages that induce the mechanisms express both FcγRI and FcγRIIIa, including FcγRIIa on the surface, for effective removal of viruses and infected cells, it is essential to maintain a binding capacity to other activating FcγRs while improving the affinity with FcγRIIa expressed on the surface of macrophages. In addition, since the higher the ratio (A/I ratio) between capacity (A) to bind to activating FcγR and capacity (I) to bind to inhibiting FcγRIIb of the Fc domain of the antibody, the better ADCC and ADCP induction ability is shown, it is very urgent to selectively increase the binding capacity of the activating receptor compared to the binding capacity of FcγRIIb, an inhibiting receptor (Boruchov et al, J Clin Invest, 115 (10): 2914-23, 2005). However, due to a problem that FcγRs have high structural homology with each other, efforts to increase the A/I ratio by introducing genetic mutations into antibodies have not reaped the great benefits.

Meanwhile, it has been recently found that neutrophils, one of the immune cells of a patient, play an important role in the treatment and progression of cancer, and much research has been conducted on treatments using these neutrophils. Neutrophil cancer cell death by antibody drugs occurs through binding to FcγRIIa expressed on the surface of the neutrophils. At this time, FcγRIIIb, another receptor expressed in the neutrophils, has no cell signaling domain, and thus when bound to FcγRIIIb, cancer cell killing is not induced. Therefore, for an effective anticancer effect by neutrophils, it is necessary to develop variants that increase a binding capacity to FcγRIIa without increasing a binding capacity to FcγRIIIb in the antibody Fc domain.

It has been reported that the half-life and persistence of immunoglobulins (antibodies) in the blood/in vivo are largely dependent on the binding of Fc to a neonatal Fc receptor (FcRn), one of IgG binding ligands. It has been reported that the FcRn is mainly expressed in endothelial cells and epithelial cells, and binds to the CH₂—CH₃ boundary region of the antibody Fc domain to maintain homeostasis of antibody concentration in the body and increase the half-life of antibodies in the blood through a recycling process to move into the cells and then be released into the plasma. Specifically, the Fc fragment of immunoglobulin is taken up by endothelial cells through non-specific cellular uptake and then introduced into acidic endosomes. The FcRn binds to immunoglobulin under acidic pH (<6.5) in endosome and releases the immunoglobulin under basic pH (>7.4) in the bloodstream. Therefore, the FcRn recovers the immunoglobulins from the lysosomal degradation pathway. When the serum immunoglobulin level decreases, the amount of immunoglobulins may be increased by using more FcRn molecules for immunoglobulin binding. On the contrary, when the serum immunoglobulin level increases, the FcRn is saturated to increase the proportion of cellularly taken immunoglobulins to be degraded (Ghetie and Ward, Annu. Rev. Immunol. 18:739-766, 2000). In other words, the blood half-life and persistence of the antibody largely depend on the binding between the Fc domain of the antibody and the neonatal Fc receptor (FcRn) as one of the IgG binding ligands. The Fc domain of the antibody, which is responsible for collecting immune leukocytes or serum complement molecules so as to remove damaged cells such as cancer cells or infected cells, is a region between the Cγ2 and Cγ3 domains, and mediates interaction with the neonatal receptor FcRn, whose binding recycles endocytosed antibodies from endosomes to the bloodstream (Raghavan et al., 1996, Annu Rev Cell Dev Biol 12:181-220; Ghetie et al., 2000, Annu Rev Immunol 18:739-766). This process is caused by the large size of the full-length molecule and associated with the inhibition of kidney filtration to have a favorable antibody serum half-life in the range of 1 to 3 weeks. In addition, the binding of Fc to FcRn also plays an important role in antibody transport. Therefore, the Fc domain plays an essential role in maintaining prolonged serum persistence by circulating antibodies through intracellular trafficking and recycling mechanisms, and thus in order to improve the blood half-life of the antibody, the pH-dependent binding capacity to FcRn needs to be improved.

### DISCLOSURE

### Technical Problem

An object of the present disclosure is to provide a novel human antibody Fc domain variant.

Another object of the present disclosure is to provide an antibody specific for an Fc gamma receptor or an immunologically active fragment thereof.

Yet another object of the present disclosure is to provide a nucleic acid molecule encoding a novel human antibody Fc domain variant, a vector including the nucleic acid molecule, and a host cell including the vector.

Still another object of the present disclosure is to provide a bioactive polypeptide conjugate with an increased in vivo half-life.

Still another object of the present disclosure is to provide a pharmaceutical composition for treating or preventing cancer.

Still another object of the present disclosure is to provide a method for preparing a human antibody Fc domain variant.

Still another object of the present disclosure is to provide a method for preparing an antibody specific for an Fc gamma receptor.

Still another object of the present disclosure is to provide a use for preparing an antibody therapeutic agent.

Still another object of the present disclosure is to provide a use for preventing or treating cancer.

Still another object of the present disclosure is to provide a method for treating cancer.

### Technical Solution

In order to solve the problems, an aspect of the present disclosure provides a novel human antibody Fc domain variant with an increased in vivo half-life and increased selective binding to a specific Fc gamma receptor.

Another aspect of the present disclosure provides an antibody including the novel human antibody Fc domain variant or an immunologically active fragment thereof.

Yet another aspect of the present disclosure provides a bioactive polypeptide conjugate including the novel human antibody Fc domain variant.

Still another aspect of the present disclosure provides a nucleic acid molecule encoding the Fc domain variant, or the antibody or immunologically active fragment thereof, a vector including the nucleic acid molecule, and a host cell including the vector.

Still another aspect of the present disclosure provides a pharmaceutical composition for treating or preventing cancer including the Fc domain variant, the bioactive polypeptide conjugate, or the antibody or immunologically active fragment thereof as an active ingredient.

Still another aspect of the present disclosure provides a method for preparing a human antibody Fc domain variant.

Still another aspect of the present disclosure provides a method for preparing an antibody specific to an Fc gamma receptor.

Still another aspect of the present disclosure provides a use of the Fc domain variant, the antibody or the immunologically active fragment thereof, or the bioactive polypeptide conjugate for use in the preparation of the antibody therapeutic agent.

Still another aspect of the present disclosure provides a use of the Fc domain variant, the antibody or the immunologically active fragment thereof, or the bioactive polypeptide conjugate for use in the prevention or treatment of cancer.

Still another aspect of the present disclosure provides a method for treating cancer comprising administering the Fc domain variant, the antibody or the immunologically active fragment thereof, or the bioactive polypeptide conjugate in a pharmaceutically effective amount to a subject with cancer.

### Advantageous Effects

According to the present disclosure, compared to a wild-type human antibody Fc domain and conventional antibodies approved as antibody therapeutic agents, novel human antibody Fc domain variants of the present disclosure have a lower capacity to bind to immune-inhibiting receptors FcγRIIb and have a higher capacity to bind to immune-activating receptor FcγRIIa and FcγRIIIb (increased A/I ratio), thereby having a remarkably improved effector function and having maximized half-life in blood in which excellent pH-selective FcRn binding and unbinding capacity is exhibited, and thus bind to numerous peptide drug therapeutics having short half-life and retention time in the body so that long-term drug efficacy through increased blood half-life can be exhibited, and can maximize the immune mechanism of therapeutic protein drugs so as to be effectively used as an improved antibody drug.

### DESCRIPTION OF DRAWINGS

FIG. 1 is a diagram illustrating results of SDS-PAGE analysis after expression and purification of FcγRIIa-131H-streptavidin-His, hFcγRIIa-131R-streptavidin-His, hFcγRIIb-streptavidin-His, hFcFcγRIIa-131H-GST, hFcFcγRIIa-131R-GST, hFcFcγRIIb-GST and hFcRn-GST.

FIG. 2 is a diagram illustrating the concentration and gating strategy of Alexa647 conjugated tetrameric FcγRIIa, non-fluorescent tetrameric FcγRIIb, and Alexa488 conjugated Protein A for each round of sorting for a yeast display library using FACS.

FIG. 3 is a diagram illustrating results of FACS analysis of (A) fluorescence intensities by FcγRIIa-Alexa647 binding of each round of Fc sub-library screened through a Saccharomyces cerevisiae cell wall display, and (B) FcγRIIa-Alexa647 binding fluorescence intensities (i.e., FcγRIIa selective binding intensity) in a state masked by non-fluorescent FcγRIIb.

FIG. 4 is a diagram illustrating results of SDS-PAGE analysis after expression and purification of Trastuzumab-Fc variants including amino acid regression Fc variants of WHFc5.

FIG. 5 is a diagram illustrating results of ELISA analysis for binding capacities to FcγRs of Trastuzumab-Fc variants including amino acid regression Fc variants of WHFc5.

FIG. 6 is a diagram illustrating results of SDS-PAGE analysis after expression and purification of Trastuzumab-Fc variants WHFc25-1, WHFc25-2 and WHFc25-3 with individual amino acid substitution combinations.

FIG. 7 is a diagram illustrating results of ELISA analysis for binding capacities to FcRn at pH 6.0 and 7.4 of Trastuzumab-Fc variants WHFc25-1, WHFc25-2 and WHFc25-3 with individual amino acid substitution combinations:
WT: Trastuzumab wild-type; and
DEA: Conventional Fc variant.

FIG. 8 is a diagram illustrating results of ELISA analysis for binding capacities to FcγRIIa of Trastuzumab-Fc variants WHFc25-1, WHFc25-2 and WHFc25-3 with individual amino acid substitution combinations:
WT: Trastuzumab wild-type; and
DEA: Conventional Fc variant.

FIG. 9 is a diagram illustrating results of ELISA analysis for binding capacities to FcγRIIIb of Trastuzumab-Fc variants WHFc25-1, WHFc25-2 and WHFc25-3 with individual amino acid substitution combinations:
WT: Trastuzumab wild-type; and
DEA: Conventional Fc variant.

FIG. 10 is a diagram illustrating results of analyzing an ADCC effect on neutrophils of Trastuzumab-Fc variants WHFc25-1, WHFc25-2 and WHFc25-3 with individual amino acid substitution combinations:
WT: Trastuzumab wild-type; and
DEA: Conventional Fc variant.

FIG. 11 is a diagram illustrating results of analyzing the macrophage ADCP efficiency of a Trastuzumab-Fc variant WHFc25-3 with individual amino acid substitution combinations:
WT: Trastuzumab wild-type;
GA: Convention Fc variant (G236A); and
DEA: Conventional Fc variant.

FIG. 12 is a diagram confirming a half-life in blood of Trastuzumab-Fc variants WHFc25-2 and WHFc25-3 with individual amino acid substitution combinations:
WT: Trastuzumab wild-type;
DEA: Conventional Fc variant; and
PFc29: Prior Fc variant of the present disclosure (positive control group).

FIG. 13 is a diagram of analyzing thermal stability of Trastuzumab-Fc variants WHFc25-1, WHFc25-2 and WHFc25-3 with individual amino acid substitution combinations:
WT: Trastuzumab wild-type;
DEA: Conventional Fc variant; and
PFc29: Prior Fc variant of the present disclosure (positive control group).

### BEST MODE OF THE INVENTION

Hereinafter, embodiments of the present disclosure will be described in detail with reference to the accompanying drawings. However, the following embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express preferred embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

All technical terms used in the present disclosure, unless otherwise defined, have the meaning as commonly understood by those skilled in the related art of the present disclosure. In addition, although preferred methods and samples are described herein, similar or equivalent methods and samples thereto are also included in the scope of the present disclosure. The contents of all publications disclosed as references in this specification are incorporated in the present disclosure.

Throughout the present specification, general one-letter or three-letter codes for naturally existing amino acids are used, and generally allowed three-letter codes for other amino acids, such as α-aminoisobutyric acid (Aib) and N-methylglycine (Sar) are also used. The amino acids mentioned herein as abbreviations are also described as follows according to the IUPAC-IUB nomenclature.

Alanine: A; Arginine: R; Asparagine: N; Aspartic acid: D; Cysteine: C; Glutamic acid: E; Glutamine: Q; Glycine: G; Histidine: H; Isoleucine: I; Leucine: L; Lysine: K; Methionine: M; Phenylalanine: F; Proline: P; Serine: S; Threonine: T; Tryptophan: W; Tyrosine: Y; and Valine: V.

In one aspect, the present disclosure relates to a human antibody Fc domain variant in which amino acids at positions 231 and 355 numbered according to the Kabat numbering system in a wide-type human antibody Fc domain are substituted with sequences different from wild-type amino acids.

In one embodiment, in the human antibody Fc domain variant of the present disclosure, one or more positions selected from the group consisting of amino acids at positions 231, 236, 311, 355, 396 and 428 may be substituted with sequences different from the wild type amino acids.

In one embodiment, the wild-type human antibody Fc domain may consist of an amino acid sequence represented by SEQ ID NO: 7, which may be encoded with a nucleic acid molecule represented by SEQ ID NO: 8.

In one embodiment, the human antibody Fc domain variant of the present disclosure may include one or more amino acid substitutions selected from the group consisting of A231V, G236A, Q311R, R355L, P396L and M428L.

In one embodiment, the human antibody Fc domain variant of the present disclosure may be a human antibody Fc domain variant WHFc25-1 including amino acid substitutions of A231V, G236A, Q311R, P396L and M428L, and the human antibody Fc domain variant WHFc25-1 may include an amino acid sequence represented by SEQ ID NO: 1, which may be encoded by a nucleic acid molecule including a nucleotide sequence represented by SEQ ID NO: 2.

In one embodiment, the human antibody Fc domain variant of the present disclosure may be a human antibody Fc domain variant WHFc25-2 including amino acid substitutions of G236A, Q311R, R355L, P396L and M428L, and the human antibody Fc domain variant WHFc25-2 may include an amino acid sequence represented by SEQ ID NO: 3, which may be encoded by a nucleic acid molecule including a nucleotide sequence represented by SEQ ID NO: 4.

In one embodiment, the human antibody Fc domain variant of the present disclosure may be a human antibody Fc domain variant WHFc25-3 including amino acid substitutions of A231V, G236A, Q311R, R355L, P396L and M428L, and the human antibody Fc domain variant WHFc25-3 may include an amino acid sequence represented by SEQ ID NO: 5, which may be encoded by a nucleic acid molecule including a nucleotide sequence represented by SEQ ID NO: 6.

In one embodiment, the human antibody Fc domain variant of the present disclosure may have an improved binding capacity to FcγRIIa compared to the wild-type human antibody Fc domain.

In one embodiment, the human antibody Fc domain variant of the present disclosure may have a selective binding capacity to FcγRIIa relative to human FcγRIIb compared to the wild-type Fc domain.

In one embodiment, the human antibody Fc domain variant of the present disclosure may have a selective binding capacity to FcγRIIa relative to FcγRIIIb compared to the wild-type human antibody Fc domain.

In one embodiment, the human antibody Fc domain variant of the present disclosure may have a significantly improved capacity to bind to an activating receptor, FcγRIIa, compared to the wild-type Fc domain, and have an improved A/I ratio compared to the wild-type, thereby selectively improving binding to human FcγRIIa compared to human FcγRIIb. In one embodiment, the human antibody Fc domain variant of the present disclosure may improve the ability to induce antibody-dependent cell-mediated phagocytosis (ADCP) compared to the wild-type human antibody Fc domain.

FcγRIIIb is a receptor that is generally expressed only in neutrophils, and since FcγRIIIb has no cell signaling domain, immune cells are not activated by an antibody binding thereto, and thus ADCC by neutrophils is improved only when selectively binding to FcγRIIa. Therefore, the Fc domain variant of the present disclosure has the effect of improving ADCC by neutrophils by selectively improving binding to human FcγRIIa compared to FcγRIIIb.

In one embodiment, the human antibody Fc domain variant of the present disclosure may have a reduced or maintained the binding capacity to FcγRIIIb (CD16b) compared to the wild-type Fc domain, whereas the binding capacity to FcγRIIa may be significantly improved to selectively improve the binding to human FcγRIIa compared to FcγRIIIb. In one embodiment, the human antibody Fc domain variant of the present disclosure may have improved ADCC compared to the wild-type human antibody Fc domain.

In one embodiment, the human antibody Fc domain variant of the present disclosure may have an improved effector function compared to the wild-type human antibody Fc domain. The effector function may be selected from the group consisting of antibody-dependent cellular cytotoxicity (ADCC), antibody-dependent cell-mediated phagocytosis (ADCP), C1q-binding, complement activation, complement dependent cytotoxicity (CDC), Fc-receptor binding including Fc-gamma receptor binding, protein A-binding, protein G-binding, complement dependent cell-mediated cytotoxicity (CDCC), complement-enhanced cytotoxicity, opsonization, Fc-containing polypeptide internalization, target downmodulation, ADC uptake, induction of apoptosis, cell death, cell cycle arrest, and any combination thereof.

In one embodiment, the human antibody Fc domain variant of the present disclosure may improve the ability to induce antibody-dependent cell-mediated phagocytosis (ADCP) compared to the wild-type human antibody Fc domain.

In one embodiment, the human antibody Fc domain variant of the present disclosure may improve the ability to induce antibody-dependent cell-mediated cytotoxicity (ADCC) compared to the wild-type human antibody Fc domain, and is more preferably ADCC by neutrophils.

In one embodiment, the human antibody Fc domain variant of the present disclosure may exhibit low binding affinity to FcRn at pH 7.0 to 7.8 compared to the wild-type human antibody Fc domain, and may be pH sensitive that exhibits high binding affinity to FcRn at pH 5.6 to 6.5 compared to the wild-type human antibody Fc domain.

In one embodiment, the Fc variant of the present disclosure may exhibit high binding affinity to FcRn compared to the wild-type immunoglobulin Fc domain at pH 5.6 to 6.5, and may be under weak acidic conditions in endosomes, and pH 5.8 to 6.0. The pH-sensitive Fc variant of the present disclosure may have increased binding affinity to FcRn in the pH range of 10% or more, 20% or more, 30% or more, 40% or more, 50% or more, 60% or more, 70% or more, 80% or more, 90% or more, or 100% or more compared to the wild-type Fc domain, or 2 times or more, 3 times or more, 4 times or more, 5 times or more, 6 times or more, 7 times or more, 8 times or more, 9 times or more, 10 times or more, 20 times or more, 30 times or more, 40 times or more, 50 times or more, 60 times or more, 70 times or more, 80 times or more, 90 times or more, or 100 times or more compared to the wild-type Fc domain.

In one embodiment, the Fc variant of the present disclosure may exhibit low binding affinity to FcRn compared to the wild-type immunoglobulin Fc domain at pH 7.0 to 7.8, and may be under a normal pH range of blood and pH 7.2 to 7.6. The degree of unbinding (dissociation) of the Fc variant of the present disclosure from FcRn in the above pH range may be the same or not be substantially changed compared to the wild-type Fc domain.

In one embodiment, the human antibody Fc domain variant of the present disclosure may have an increased in vivo half-life compared to the wild-type human antibody Fc domain.

In one embodiment, the human antibody Fc domain variant of the present disclosure may have an increased in vivo half-life in blood compared to the wild-type human antibody Fc domain.

In one embodiment, the half-life of the human antibody Fc domain variant of the present disclosure may be increased at least 10%, at least 20%, at least 30%, at least 40%, at least 50%, at least 60%, at least 70%, at least 80%, at least 90%, or at least 100% compared to the wild-type human antibody Fc domain, or increased at least 2 times, at least 3 times, at least 4 times, at least 5 times, at least 6 times, at least 7 times, at least 8 times, at least 9 times or at least 10 times compared to the wild type Fc domain.

In one embodiment, the human antibody (immunoglobulin) may be IgA, IgM, IgE, IgD or IgG, or modifications thereof, and may be IgG1, IgG2, IgG3 or IgG4, preferably an anti-HER2 antibody, and more preferably Trastuzumab. Papain degradation of the antibody forms two Fab domains and one Fc domain, and in a human IgG molecule, the Fc domain is generated by papain degradation of the N-terminus at Cys 226 (Deisenhofer, Biochemistry 20:2361-2370, 1981).

In the present disclosure, variants including amino acid mutations in the human antibody Fc domain of the present disclosure are defined according to the amino acid modifications constituting an Fc domain of a parent antibody, and conventional antibody numbering is followed by the EU index by Kabat (Kabat et al., Sequence of proteins of immunological interest, 5th Ed., United States Public Health Service, National Institutes of Health, Bethesda, 1991).

As used herein, the term “Fc domain variant” may be used interchangeably with the “Fc variant”.

As used herein, the term “wild-type polypeptide” refers to an unmodified polypeptide that is modified later to produce a derivative. The wild-type polypeptide may be a polypeptide found in nature, or a derivative or manipulation of the polypeptide found in nature. The wild-type polypeptide may refer to a polypeptide itself, a composition including the wild-type polypeptide, or an amino acid sequence encoding the same. Accordingly, as used herein, the term “wild-type antibody” refers to an unmodified antibody polypeptide in which amino acid residues are modified to produce a derivative. Interchangeably with the term, the “parent antibody” may be used to refer to an unmodified antibody polypeptide into which amino acid modifications are introduced to produce a derivative.

As used herein, the term “amino acid modification/mutation” refers to substitution, insertion and/or deletion, preferably substitution of amino acids in a polypeptide sequence. As used herein, the term “amino acid substitution” or “substitution” means that an amino acid at a specific position in the polypeptide sequence of the wild-type human antibody Fc domain is replaced with another amino acid. For example, an Fc variant including Q311R substitution means that glutamine, an amino acid residue at position 311 in the amino acid sequence of the wild-type antibody Fc domain, is replaced with arginine.

As used herein, the term “Fc variant” means including a modification of one or more amino acid residues compared to the wild-type antibody Fc domain.

The Fc variant of the present disclosure includes one or more amino acid modifications compared to the wild-type antibody Fc domain (region or fragment), resulting in a difference in amino acid sequence. The amino acid sequence of the Fc variant according to the present disclosure is substantially homologous to the amino acid sequence of the wild-type antibody Fc domain. For example, the amino acid sequence of the Fc variant according to the present disclosure has about 80% or more, preferably about 90% or more, and most preferably about 95% or more homology compared to the amino acid sequence of the wild-type antibody Fc domain. The amino acid modifications may be performed genetically using molecular biological methods, or also performed using enzymatic or chemical methods.

The Fc variants of the present disclosure may be prepared by any method known in the art. In one embodiment, the human antibody Fc variant according to the present disclosure is used to form a nucleic acid in which a polypeptide sequence including a specific amino acid modification is encoded and then, if desired, cloned into a host cell, expressed, and assayed. Various methods therefor are described in the literature (Molecular Cloning-A Laboratory Manual, 3rd Ed., Maniatis, Cold Spring Harbor Laboratory Press, New York, 2001; Current Protocols in Molecular Biology, John Wiley & Sons).

The nucleic acid encoding the Fc variant according to the present disclosure may be inserted into an expression vector for protein expression. The expression vector generally includes a protein operably linked, i.e., functionally related to a control or regulatory sequence, a selectable marker, an optional fusion partner, and/or an additional element. Under appropriate conditions, the Fc variant according to the present disclosure may be produced by a method of inducing the protein expression by incubating a host cell transformed with a nucleic acid, preferably a nucleic acid-containing expression vector encoding the Fc variant according to the present disclosure. A variety of suitable host cells including mammalian cells, bacteria, insect cells, and yeast may be used, but are not limited thereto. Methods for introducing exogenous nucleic acid into a host cell are known in the art and will vary depending on a host cell to be used. Preferably, the Fc variant according to the present disclosure is produced using E. coli, which has low production cost and high industrial value, as a host cell.

Accordingly, the scope of the present disclosure includes a method for preparing an Fc variant including incubating a host cell into which a nucleic acid encoding an Fc variant has been introduced, under conditions suitable for protein expression; and purifying or isolating the Fc variant expressed from the host cell.

As used herein, the term “FcRn” or “neonatal Fc receptor” refers to a protein that binds to an IgG antibody Fc domain, which is at least partially encoded by an FcRn gene. The FcRn may be derived from any organism, including humans, mice, rats, rabbits, and monkeys, but is not limited thereto. As known in the art, a functional FcRn protein includes two polypeptides, often referred to as light and heavy chains. The light chain is β-2-microglobulin, and the heavy chain is encoded by the FcRn gene. Unless otherwise stated herein, the FcRn or one FcRn protein refers to a complex of the FcRn heavy chain and the β-2-microglobulin.

In one aspect, the present disclosure relates to an antibody specific for an Fc gamma receptor including the Fc domain variant of the present disclosure or an immunologically active fragment thereof.

In one embodiment, the antibody of the present disclosure may have an improved binding capacity to the Fc gamma receptors.

In one embodiment, the antibody of the present disclosure may have an increased in vivo half-life compared to the wild-type human antibody.

In one embodiment, the antibody may be a polyclonal antibody, a monoclonal antibody, a minibody, a domain antibody, a bispecific antibody, an antibody mimetic, a chimeric antibody, an antibody conjugate, a human antibody, or a humanized antibody. The immunologically active fragment may be Fab, Fd, Fab′, dAb, F(ab′), F(ab′)2, a single chain fragment variable (scFv), Fv, a single chain antibody, an Fv dimer, a complementarity determining region fragment, or a diabody.

In one embodiment, the antibody including the Fc domain variant of the present disclosure or the immunologically active fragment thereof may increase an effector action, and has an improved binding capacity to FcγRIIa compared to the binding capacity to FcγRIIb compared to the wild-type Fc domain to have high FcγRIIa binding selectivity and a high A/I ratio, thereby increasing antibody-dependent cell-mediated phagocytosis (ADCP).

In the present disclosure, the A/I ratio is a ratio (A/I ratio) between the ability (A) of the Fc domain of the antibody to bind to activating FcγR and the ability (I) to bind to inhibitory FcγRIIb, and as the A/I ratio is increased, excellent ability to induce ADCC and ADCP is shown, so that it is important to selectively increase the binding capacity to the activating receptor compared to the binding capacity to FcγRIIb, which is an inhibiting receptor.

In one embodiment, the antibody including the Fc domain variant of the present disclosure or the immunologically active fragment thereof may increase an effector action, and has an improved binding capacity to FcγRIIa compared to the binding capacity to FcγRIIIb compared to the wild-type Fc domain to have high FcγRIIa binding selectivity, thereby increasing ADCC by neutrophils.

In the present disclosure, since FcγRIIIb, which is a receptor that is generally expressed only in neutrophils, has no cell signaling domain, immune cells are not activated by an antibody binding thereto, and thus ADCC by neutrophils is improved only when selectively binding to FcγRIIa. Therefore, it is important to selectively increase the ratio of the binding capacity to FcγRIIa compared to the binding capacity to FcγRIIIb.

The antibody may be isolated or purified by various methods known in the art. A standard purification method includes chromatographic techniques, electrophoresis, immunology, precipitation, dialysis, filtration, concentration, and chromatofocusing techniques. As known in the art, various natural proteins such as bacterial proteins A, G, and L are bound to the antibody and these proteins may be used for purification. Often, purification by specific fusion partners may be enabled.

The antibody includes functional fragments of an antibody molecule as well as a whole antibody form. The whole antibody has a structure having two full-length light chains and two full-length heavy chains, and each light chain is linked to the heavy chain by disulfide bonds. The functional fragment of the antibody molecule refers to a fragment having an antigen-binding function. Examples of the antibody fragment include (i) a Fab fragment consisting of a light chain variable region VL, a heavy chain variable region VH, a light chain constant region CL, and a first heavy chain constant region CH1; (ii) a Fd fragment consisting of VH and CH1 domains; (iii) an Fv fragment consisting of VL and VH domains of a single antibody; (iv) a dAb fragment consisting of a VH domain (Ward E S et al., Nature 341:544-546 (1989)); (v) an isolated CDR region; (vi) a F(ab′)2 fragment, which is a bivalent fragment including two linked Fab fragments; (vii) a single-chain Fv molecule (scFv) in which the VH domain and the VL domain are linked to each other by a peptide linker to form an antigen-binding domain; (viii) a bispecific single-chain Fv dimer (PCT/US92/09965); and (ix) a diabody which is a multivalent or multispecific fragment produced by gene fusion (WO94/13804).

The antibody of the present disclosure or the immunologically active fragment thereof may be selected from the group consisting of animal-derived antibodies, chimeric antibodies, humanized antibodies, human antibodies, and immunologically active fragments thereof. The antibody may be produced recombinantly or synthetically.

The antibody or the immunologically active fragment thereof may be isolated from a living body (not present in a living body) or may non-naturally occur, and for example, may be synthetically or recombinantly produced.

As used herein, the “antibody” refers to a substance produced by stimulation of an antigen in the immune system, and the type thereof is not particularly limited, and may be obtained naturally or non-naturally (e.g., synthetically or recombinantly). The antibody is advantageous for mass expression and production because of being very stable in vivo as well as ex vivo and having a long half-life. In addition, since the antibody essentially has a dimer structure, avidity is very high. An intact antibody has a structure having two full-length light chains and two full-length heavy chains, and each light chain is linked to the heavy chain by disulfide bonds. The constant region of the antibody is divided into a heavy chain constant region and a light chain constant region, and the heavy chain constant region has gamma (γ), mu (μ), alpha (α), delta (δ), and epsilon (ε) types, and subclasses include gamma 1 (γ1), gamma 2 (γ2), gamma 3 (γ3), gamma 4 (γ4), alpha 1 (α1) and alpha 2 (α2). The light chain constant region has kappa (κ) and lambda (λ) types.

As used herein, the term “heavy chain” is interpreted as meaning including all of a full-length heavy chain including a variable region domain VH including an amino acid sequence having a sufficient variable region sequence to impart specificity to an antigen, three constant region domains C_{H}1, C_{H}2 and C_{H}3 and hinges, and fragments thereof. In addition, the term “light chain” is interpreted as meaning including all of a full-length light chain including a variable region domain V_{L} including an amino acid sequence having a sufficient variable region sequence to impart specificity to an antigen and a constant region domain C_{L}, and fragments thereof.

As used herein, the term “Fc domain”, “Fc fragment”, or “Fc domain” forms an antibody with a Fab domain/fragment, and the Fab domain/fragment consists of a light chain variable region V_{L} and a heavy chain variable region V_{H}, a light chain constant region C_{L}, and a first heavy chain constant region C_{H}1, and the Fc domain/fragment consists of a second constant region C_{H}2 and a third constant region C_{H}3 of the heavy chain.

In one aspect, the present disclosure relates to a nucleic acid molecule encoding the Fc domain variant of the present disclosure, or an antibody including the Fc domain variant or an immunologically active fragment thereof.

In one embodiment, the nucleic acid molecule encoding the Fc variant according to the present disclosure may include a nucleotide sequence represented by SEQ ID NO: 2, 4, or 6.

In one aspect, the present disclosure relates to a vector including the nucleic acid molecule and a host cell including the vector.

The nucleic acid molecule of the present disclosure may be isolated or recombinant, and includes not only DNA and RNA in single-stranded and double-stranded forms, but also complementary sequences corresponding thereto. The isolated nucleic acid is a nucleic acid that is isolated from surrounding genetic sequences present in the genome of a subject from which the nucleic acid is isolated, in the case of a nucleic acid isolated from a naturally occurring source. In the case of a nucleic acid synthesized enzymatically or chemically from a template, such as a PCR product, a cDNA molecule, or an oligonucleotide, the nucleic acid produced from such a procedure may be understood as an isolated nucleic acid molecule. The isolated nucleic acid molecule refers to a nucleic acid molecule in the form of a separate fragment or as a component of a larger nucleic acid construct. The nucleic acid is operably linked when disposed in a functional relationship with another nucleic acid sequence. For example, DNA of a pre-sequence or secretory leader is expressed as a preprotein in the form before the polypeptide is secreted to be operably linked to DNA of the polypeptide, and a promoter or enhancer is operably linked to a coding sequence when affecting the transcription of the polypeptide sequence, or a ribosome binding domain is operably linked to a coding sequence when disposed to promote the translation. In general, the operably linked means that DNA sequences to be linked are located contiguously, and that the secretory leader exists contiguously in the same leading frame. However, the enhancer needs not to be contiguously located. The linkage is achieved by ligation at a convenient restriction enzyme site. When there is no such a site, synthetic oligonucleotide adapters or linkers are used according to conventional methods.

In the isolated nucleic acid molecule encoding the Fc domain variant of the present disclosure, or the antibody including the Fc domain variant or immunologically active fragment thereof, due to the degeneracy of codons or in consideration of codons preferred in an organism in which the protein is to be expressed, it will be well understood by those skilled in the art that various modifications may be made to a coding region within a range without changing the amino acid sequence of the Fc domain variant of the present disclosure, or the antibody including the Fc domain variant or immunologically active fragment thereof to be expressed from the coding region, various modifications or changes may be made within a range without affecting the expression of the gene even in parts other than the coding region, and such modified genes are also included within the scope of the present disclosure. That is, as long as the nucleic acid molecule of the present disclosure encodes a protein having equivalent activity thereto, one or more nucleotides may be mutated by substitution, deletion, insertion, or a combination thereof, which are also included in the scope of the present disclosure. The sequence of such a nucleic acid molecule may be single- or double-stranded, and may be a DNA molecule or an RNA (mRNA) molecule.

The isolated nucleic acid molecule encoding the Fc domain variant of the present disclosure, or the antibody including the Fc domain variant or immunologically active fragment thereof may be inserted into an expression vector for protein expression. The expression vector generally includes a protein operably linked, i.e., functionally related to a control or regulatory sequence, a selectable marker, an optional fusion partner, and/or an additional element. In appropriate conditions, the Fc domain variant of the present disclosure, or the antibody including the Fc domain variant or immunologically active fragment thereof may be produced by a method for inducing the protein expression by incubating a host cell transformed with a nucleic acid, preferably an expression vector including an isolated nucleic acid molecule encoding the Fc domain variant of the present disclosure, the antibody including the Fc domain variant or the immunologically active fragment thereof. A variety of suitable host cells including mammalian cells, bacteria, insect cells, and yeast may be used, but are not limited thereto. Methods for introducing exogenous nucleic acid into a host cell are known in the art and will vary depending on a host cell to be used. Preferably, it is possible to produce E. coli, which has high industrial value due to low production cost, as a host cell.

The vector of the present disclosure may include a plasmid vector, a cosmid vector, a bacteriophage vector, a viral vector, etc., but is not limited thereto. The suitable vector includes a signal sequence or a leader sequence for membrane targeting or secretion in addition to expression regulatory elements such as a promoter, an operator, an initiation codon, a termination codon, a polyadenylation signal, and an enhancer and may be variously produced according to the purpose. The promoter of the vector may be constitutive or inductive. The signal sequence may use a PhoA signal sequence, an OmpA signal sequence, etc. in the case of Escherichia sp. as a host, an α-amylase signal sequence, a subtilisin signal sequence, etc. in the case of Bacillus sp. as a host, an MFα signal sequence, a SUC2 signal sequence, etc. in the case of yeast as a host, and an insulin signal sequence, an α-interferon signal sequence, an antibody molecule signal sequence, etc. in the case of an animal cell as a host, but is not limited thereto. Further, the vector may include a selective marker for selecting a host cell including a vector and a replicable expression vector includes a replication origin.

As used herein, the term “vector” refers to a vehicle into which a nucleic acid sequence may be inserted for introduction into a cell capable of replicating the nucleic acid sequence. The nucleic acid sequence may be exogenous or heterologous. The vector may include plasmids, cosmids, and viruses (e.g., bacteriophages), but is not limited thereto. Those skilled in the art may construct vectors by standard recombinant techniques (Maniatis, et al., Molecular Cloning, A Laboratory Manual, Cold Spring Harbor Press, Cold Spring Harbor, N.Y., 1988; and Ausubel et al., In: Current Protocols in Molecular Biology, John, Wiley & Sons, Inc, NY, 1994, etc.).

In one embodiment, when constructing the vector, expression regulatory sequences such as a promoter, a terminator, and an enhancer, sequences for membrane targeting or secretion, etc. may be appropriately selected according to a type of host cell to produce the Fc domain variant, or the antibody including the Fc domain variant or immunologically active fragment thereof and may be variously combined depending on a purpose.

As used herein, the term “expression vector” refers to a vector including a nucleic acid sequence encoding at least a portion of a gene product to be transcribed. In some cases, the RNA molecule is then translated into a protein, a polypeptide, or a peptide. The expression vector may include various regulatory sequences. In addition to regulatory sequences that regulate transcription and translation, vectors and expression vectors may also include nucleic acid sequences that provide other functions.

As used herein, the term “host cell” includes eukaryotes and prokaryotes, and refers to any transformable organism capable of replicating the vector or expressing a gene encoded by the vector. The host cell may be transfected or transformed by the vector, which means a process in which an exogenous nucleic acid molecule is delivered or introduced into the host cell.

In one embodiment, the host cell may be bacteria or animal cells, the animal cell line may be a CHO cell, a HEK cell or a NSO cell, and the bacteria may be E. coli.

In one aspect, the present disclosure relates to a bioactive polypeptide conjugate having an increased in vivo half-life by binding the human antibody Fc domain variant of the present disclosure and a bioactive polypeptide.

In one embodiment, the bioactive polypeptide may be selected from the group consisting of human growth hormone, growth hormone-releasing hormone, growth hormone-releasing peptide, interferon, colony-stimulating factor, interleukin, interleukin soluble receptor, TNF soluble receptor, glucocerebrosidase, macrophage activator, macrophage peptide, B-cell factor, T-cell factor, protein A, allergy suppressor, necrosis glycoprotein, immunotoxin, lymphotoxin, tumor necrosis factor, tumor suppressor, metastasis growth factor, alpha-1 antitrypsin, albumin, apolipoprotein-E, erythropoietin, hyperglycosylated erythropoietin, blood factor VII, blood factor VIII, blood factor IX, plasminogen activator, urokinase, streptokinase, protein C, C-reactive protein, renin inhibitor, collagenase inhibitor, superoxide dismutase, leptin, platelet-derived growth factor, epidermal growth factor, osteogenic growth factor, bone formation-promoting protein, calcitonin, insulin, insulin derivatives, glucagon, glucagon like peptide-1, atriopeptin, cartilage-inducing factor, connective tissue activator, follicle-stimulating hormone, luteinizing hormone, follicle-stimulating hormone-releasing hormone, nerve growth factor, parathyroid hormone, relaxin, secretin, somatomedin, insulin-like growth factor, corticosteroid, cholecystokinin, pancreatic polypeptide, gastrin-releasing peptide, corticotropin-releasing factor, thyroid-stimulating hormone, receptors, receptor antagonist, cell surface antigen, monoclonal antibody, polyclonal antibody, antibody fragments, and virus-derived vaccine antigens, and anything necessary to increase blood half-life may be used without any particular restrictions.

In one embodiment, the human antibody Fc domain variant of the present disclosure may be usefully used as a carrier to increase the in vivo half-life of a bioactive polypeptide such as a protein drug, and the bioactive polypeptide conjugate including the human antibody Fc domain variant may be used as a sustained drug formulation with a significantly increased in vivo half-life.

In one embodiment, the human antibody Fc domain variant of the present disclosure and the bioactive polypeptide may be linked by a non-peptidyl polymer, and the non-peptide polymer usable herein may be selected from the group consisting of polyethylene glycol, polypropylene glycol, copolymer of ethylene glycol and propylene glycol, polyoxyethylated polyol, polyvinyl alcohol, polysaccharide, dextran, polyvinyl ethyl ether, biodegradable polymer such as polylactic acid (PLA) and polylactic-glycolic acid (PLGA), lipid polymer, chitin, hyaluronic acid, and combinations thereof, preferably polyethylene glycol. Derivatives thereof which are already known in the art and derivatives which may be easily prepared in a technical level of the art are included in the scope of the present disclosure.

In one embodiment, an antibody drug may be bound to the bioactive polypeptide conjugate including the human antibody Fc domain variant of the present disclosure, and the antibody drug for cancer therapy may be Trastzumab, Cetuximab, Bevacizumab, Rituximab, Basiliximab, Infliximab, Ipilimumab, Pembrolizumab, Nivolumab, Atezolizumab, or Avelumab.

In the antibody therapeutic agent, a mechanism for collecting and delivering immune cells to a target antigen is one of the most important mechanisms, and since the Fc domain of the antibody plays a critical role in the collection of immune cells and antibody-dependent cell-mediated phagocytosis (ADCP), the Fc variant with increased selective binding to the Fc gamma receptor of the present disclosure is advantageous to be used as a therapeutic antibody. In particular, since the ADCP function of the antibody depends on interaction with the Fc gamma receptors (FcγR) present on the surfaces of many cells, and a type of immune cell to be collected is determined depending on which Fc receptor among five human Fc receptors the antibody binds to, attempts to modify the antibody to collect a specific cell are very important in the therapeutic field.

The present disclosure includes a method for preparing a sustained drug formulation by covalently linking the human body Fc domain variant of the present disclosure to a bioactive polypeptide through a non-peptidyl polymer.

The preparation method according to the present disclosure may include covalently linking a bioactive polypeptide and a human antibody Fc domain variant through a non-peptide polymer having a reactive group at a terminal; and isolating a conjugate in which the bioactive polypeptide, the non-peptide polymer, and the human antibody Fc domain variant are covalently linked.

In one aspect, the present disclosure relates to a pharmaceutical composition for preventing or treating cancer including the human antibody Fc domain variant of the present disclosure, the antibody including the Fc domain variant or immunologically active fragment thereof, or the bioactive polypeptide conjugate including the human antibody Fc domain variant as an active ingredient.

In one embodiment, the cancer may be any one selected from the group consisting of brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, central nervous system tumor, primary central nervous system lymphoma, spinal cord tumor, brainstem glioma and pituitary adenoma.

In one embodiment, the antibody including the Fc domain variant of the present disclosure or the immunologically active fragment thereof has high FcγRIIa binding selectivity and a high A/I ratio to increase the effector action and increase phagocytosis, so that the antibody including the Fc domain variant having FcγRIIa binding selectivity of the present disclosure or the immunologically active fragment thereof may maximize a cancer cell killing mechanism.

In one embodiment, the composition of the present disclosure may further include an immunogenic cell death inducing agent. The immunogenic cell death inducing agent may be any one or more selected from the group consisting of anthracycline anticancer agents, taxane anticancer agents, anti-EGFR antibodies, BK channel agonists, bortezomib, cardiac glycoside, cyclophosmide anticancer agents, GADD34/PP1 inhibitors, LV-tSMAC, Measles virus, bleomycin, mitoxantrone, or oxaliplatin. The anthracycline anticancer agent may be daunorubicin, doxorubicin, epirubicin, idarubicin, pixantrone, sabarubicin, or valrubicin, and the taxane anticancer agent may be paclitaxel or docetaxel.

The pharmaceutical composition for preventing or treating cancer of the present disclosure is administered together with a chemical anticancer drug (anticancer agent) and the like to increase a cancer therapy effect of conventional anticancer agents through the death effect of cancer cells. Combined administration may be performed simultaneously or sequentially with the anticancer agent. Examples of the anticancer agent include DNA alkylating agents such as mechloethamine, chlorambucil, phenylalanine, mustard, cyclophosphamide, ifosfamide, carmustine (BCNU), lomustine (CCNU), streptozotocin, busulfan, thiotepa, cisplatin, and carboplatin; anti-cancer antibiotics such as dactinomycin (actinomycin D), plicamycin, and mitomycin C; plant alkaloids such as vincristine, vinblastine, etoposide, teniposide, topotecan, and iridotecan, and the like, but are not limited thereto.

As used herein, the term “prevention” means all actions that inhibit or delay the occurrence, spread, and recurrence of cancer by administration of the pharmaceutical composition according to the present disclosure.

As used herein, the term “treatment” refers to all actions that improve or beneficially change the death of cancer cells or the symptoms of cancer by administration of the composition of the present disclosure. Those skilled in the art to which the present disclosure pertains will be able to determine the degree of improvement, enhancement and treatment by knowing the exact criteria of disease for which the composition of the present disclosure is effective by referring to data presented by the Korean Academy of Medical Sciences, etc.

As used herein, the term “therapeutically effective amount” used in combination with the active ingredient means an amount of pharmaceutically acceptable salt of the composition effective for preventing or treating a target disease, and the therapeutically effective amount of the composition of the present disclosure may vary depending on many factors, such as a method of administration, a target site, the condition of a patient, and the like. Accordingly, when used in the human body, a dose should be determined as an appropriate amount in consideration of both safety and efficiency. It is also possible to estimate the amount used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E.W. Martin ed., Remington's Pharmaceutical Sciences, 18th ed. (1990), Mack Publishing Co.

The pharmaceutical composition of the present disclosure is administered in a pharmaceutically effective amount. As used herein, the term ‘pharmaceutically effective amount’ refers to an amount enough to treat the disease at a reasonable benefit/risk ratio applicable to medical treatment and enough not to cause side effects. The effective dose level may be determined according to factors including the health condition of a patient, the type and severity of cancer, the activity of a drug, the sensitivity to a drug, a method of administration, a time of administration, a route of administration, an excretion rate, duration of treatment, and combined simultaneously used drugs, and other factors well-known in the medical field. The composition of the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

The pharmaceutical composition of the present disclosure may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc and the like. The pharmaceutically acceptable additive according to the present disclosure is preferably included in an amount of 0.1 parts by weight to 90 parts by weight based on the composition, but is not limited thereto.

The composition of the present disclosure may include a carrier, a diluent, an excipient, or a combination of two or more thereof, which are commonly used in biological agents. The pharmaceutically acceptable carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these ingredients, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using a suitable method in the art or a method disclosed in Remington's Pharmaceutical Science (Mack Publishing Company, Easton PA, 18th, 1990).

The composition of the present disclosure may be administered parenterally (e.g., applied with injectable formulations intravenously, subcutaneously, intraperitoneally or topically) or orally according to a desired method, and the range of the dose may vary depending on the body weight, age, sex, and health condition of a patient, a diet, an administration time, an administration method, an excretion rate, the severity of a disease, and the like. A daily dose of the composition according to the present disclosure is 0.0001 to 10 mg/ml, preferably 0.0001 to 5 mg/ml, and more preferably administered once to several times a day.

Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like.

In one aspect, the present disclosure relates to a method for preparing a human antibody Fc domain variant including a) incubating a host cell including a vector including a nucleic acid molecule encoding the human antibody Fc domain variant of the present disclosure; and b) recovering a polypeptide expressed by the host cell.

In one aspect, the present disclosure relates to a method for preparing an antibody specific for an Fc gamma receptor including a) incubating a host cell including a vector including a nucleic acid molecule encoding the antibody of the present disclosure or the immunologically active fragment thereof; and b) purifying the antibody expressed from the host cell.

In one embodiment, the purification of the antibody may include filtration, HPLC, anion exchange or cation exchange, high performance liquid chromatography (HPLC), affinity chromatography, or a combination thereof, preferably affinity chromatography using Protein A.

In one aspect, the present disclosure relates to a use of a human antibody Fc domain variant of the present disclosure, an antibody including the human antibody Fc domain variant or an immunologically active fragment thereof, or a bioactive polypeptide conjugate including the human antibody Fc domain variant, for use in the preparation of an antibody therapeutic agent.

In one aspect, the present disclosure relates to a use of a human antibody Fc domain variant of the present disclosure, an antibody including the human antibody Fc domain variant or an immunologically active fragment thereof, or a bioactive polypeptide conjugate including the human antibody Fc domain variant for preventing or treating cancer.

In one aspect, the present disclosure relates to a method for treating cancer including administering a human antibody Fc domain variant of the present disclosure, an antibody including the human antibody Fc domain variant or an immunologically active fragment thereof, or a bioactive polypeptide conjugate including the human antibody Fc domain variant in a pharmaceutically effective amount, to a subject with cancer.

### MODES OF THE INVENTION

Hereinafter, the present disclosure will be described in more detail with reference to the following Examples. However, the following Examples are only intended to embody the contents of the present disclosure, and the present disclosure is not limited thereto.

### Example 1. Expression and Purification of Target Substances

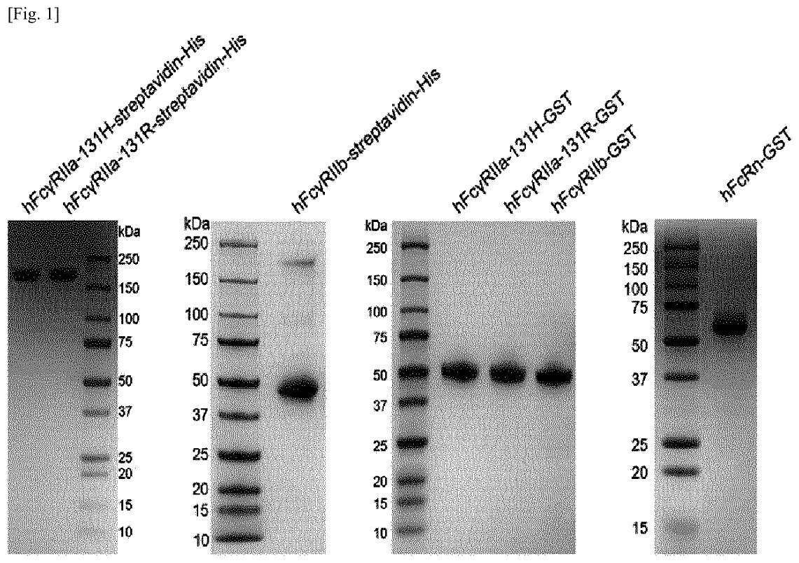

To discover Fc variants with improved binding capacity to FcγRIIa and to analyze the binding capacity of Fc variants to FcγRs and FcRn, target substances were expressed and purified. Specifically, seven expression vectors were constructed, including pMAZ-hFcγRIIa-131H-streptavidin-His, pMAZ-hFcγRIIa-131R-streptavidin-His, pMAZ-hFcγRIIb-streptavidin-His, pMAZ-hFcγRIIa-131H-GST, pMAZ-hFcγRIIa-131R-GST, pMAZ-hFcγRIIb-GST, and pMAZ-hFcRn-GST. Polyethylenimine (PEI, Polyscience, 23966) and the expression vector gene were mixed in a 4:1 ratio in 30 ml of a Freestyle 293 expression medium (Gibco, 12338-018), incubated for 20 minutes at room temperature, transfected into Expi293F animal cells incubated at a density of 2×10⁶ cells/ml, and incubated for 7 days under conditions of 37° C., 125 rpm, and 8% CO₂. After incubation, the supernatant was collected by centrifugation at 6000×g for 15 minutes, and 12.5 ml of 25×PBS was mixed with 30 ml of the culture supernatant and filtered using a 0.2 μm bottle top filter (Corning, 430513). 500 μl of Ni-NTA resin (QIAGEN, 40724) was added to a culture medium transfected with each of the filtered pMAZ-hFcγRIIa-131H-streptavidin-His, pMAZ-hFcγRIIa-131R-streptavidin-His, and pMAZ-hFcγRIIb-streptavidin-His, stirred for 16 hours at 4° C., and then packed in a disposable polypropylene column to recover the resin. The resin was washed sequentially with 100 ml of PBS (pH 7.4), 25 ml of 10 mM imidazole buffer diluted in PBS, and 25 ml of 20 mM imidazole buffer diluted in PBS, and eluted with 250 mM imidazole buffer diluted in 4 ml of PBS. In addition, 500 μl of GST resin (Incospharm, 1101-3) was added to the culture medium transfected with each of the filtered pMAZ-hFcγRIIa-131H-GST, pMAZ-hFcγRIIa-131R-GST, pMAZ-hFcγRIIb-GST, and pMAZ-hFcRn-GST, stirred at 4° C. for 16 hours, and then packed in a disposable polypropylene column to recover the resin. The resin was washed with 100 ml 1×PBS (pH 7.4) and eluted with 50 mM Tris-HCl and 10 mM GSH buffer (pH 8.0) diluted in 4 ml of 1×PBS. Thereafter, the buffer was exchanged with 1×PBS (pH 7.4) using Amicon Ultra-4 centrifugal filter units 3K (Merck Millipore, UFC800324), and as a result, it was confirmed by SDS-PAGE gel that high purity of hFcγRIIa-131H-streptavidin-His, hFcγRIIa-131R-streptavidin-His, hFcγRIIb-streptavidin-His, hFcγRIIa-131H-GST, hFcγRIIa-131R-GST, hFcγRIIb-GST and hFcRn-GST were successfully purified (FIG. 1). Among these, the purified hFcγRIIa-131H-streptavidin-His and hFcγRIIa-131R-streptavidin-His were mixed at a ratio of 1:1 and Alexa647-conjugated tetrameric FcγRIIa was prepared using Alexa Fluor™ 647 Protein Labeling Kit (Invitrogen, A20173).

### Example 2. Construction of Yeast Display Library for Sorting Fc Variants with Improved FcγRIIa Binding Capacity

To efficiently sort Fc variants with improved binding capacity to FcγRIIa, a DNA library was constructed in which mutations were introduced into an antibody Fc sequence introduced with Q311R/M428L having improved pH-dependent binding to FcRn with a probability of 1.468% of the whole Fc sequence through error-prone PCR. A yeast display library with diversity of a total of 3.25×10⁷ antibody Fc was constructed by transferring a total of 12 μg of the constructed DNA library to 400 μl of yeast sp. Saccharomyces cerevisiae competent cells through electroporation using a MicroPulser Electroporator (Bio-Rad, #1652100) along with 4 μg of a pCTCON vector encoding an Aga2 protein, a yeast cell wall anchoring protein for yeast display and a transformation selection marker gene (Trp1) and then incubating the cells in 500 ml of a tryptophan-free SD medium [Difco Yeast nitrogen base (BD, 291940) 6.7 g/l, Bacto casamino acid (BD, 223050) 5.0 g/l, Na₂HPO₄ (JUNSEI, 7558-79-4) 5.4 g/l, NaH₂PO₄·H₂O (SAMCHUN, 10049-21-5) 8.56 g/l and Glucose (DUKSAN, 50-99-7) 20 g/l] to selectively survive only the transformed yeast sp.

### Example 3. Discovery of Fc Variants with Improved Binding Capacity to FcγRIIa

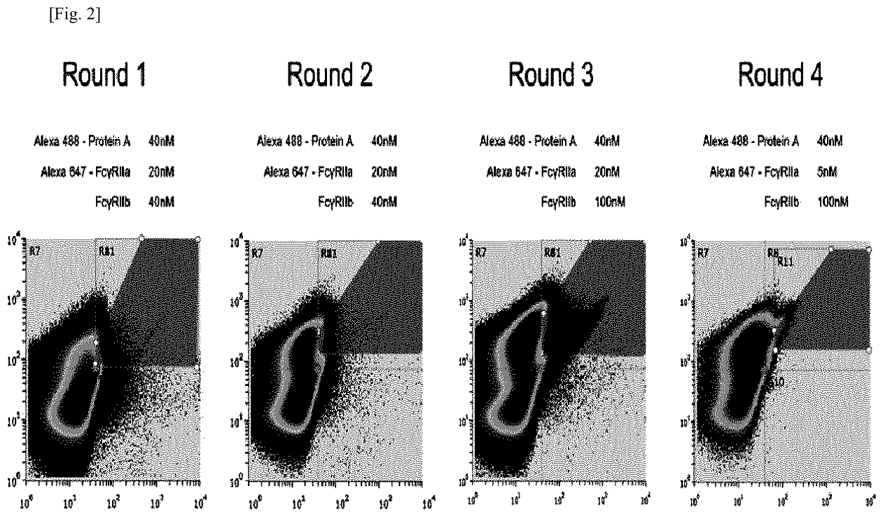

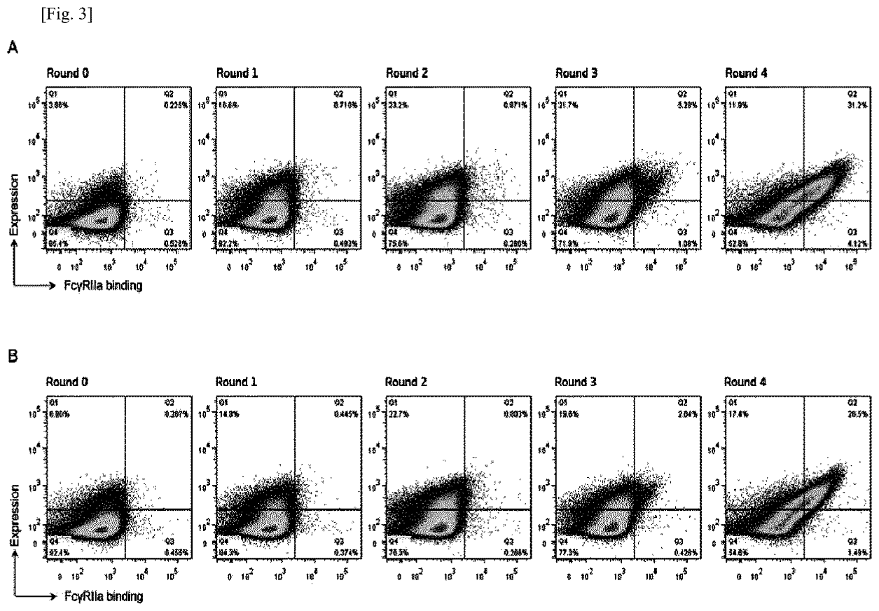

The expression levels of the displayed Fc library were improved by incubating 5.5×10⁸ cells of the constructed yeast library in 100 ml of the tryptophan-free SD medium for 16 hours at 30° C., and then incubating 7×10⁸ cells in 100 ml of a tryptophan-free SG medium [Difco Yeast nitrogen base (BD, 291940) 6.7 g/l, Bacto casamino acid (BD, 223050) 5.0 g/l, Na₂HPO₄ (JUNSEI,7558-79-4) 5.4 g/l, NaH₂PO₄·H₂O (SAMCHUN, 10049-21-5) 8.56 g/l and Galactose (Sigma-Aldrich, 59-23-4) 20 g/l] for 48 hours at 20° C. Among the incubated cells, 1×10⁸ cells were centrifuged at 14,000×g for 30 seconds at 4° C., and the supernatant was removed, and then washed with 1 ml PBSB (1×PBS, 0.1% bovine serum albumin (gibco, 30063-572)) (pH 7.4) and then 40 nM Alexa488 conjugated Protein A, 20 nM Alexa647 conjugated tetrameric FcγRIIa, and 40 nM non-fluorescent tetrameric FcγRIIb were incubated in 1 ml of diluted PBSB (pH 7.4) for 1 hour. After incubation, the cells were washed with 1 ml of PBSB (pH 7.4) and resuspended in 1 ml of PBSB (pH 7.4) to induce competitive binding of Alexa488-conjugated Protein A, Alexa647-conjugated tetrameric FcγRIIa, and non-fluorescent FcγRIIb, and then cells showing high fluorescence signals were recovered through a flow cytometer (Bio-Rad S3 sorter, Bio-Rad, #1451029). The recovered sublibraries reduced the concentration of Alexa647 conjugated tetrameric FcγRIIa or increased the concentration of non-fluorescent tetrameric FcγRIIb (FIG. 2). Through a total of four rounds of sorting, cells displaying Fc variants with an excellent binding capacity to FcγRIIa were enriched (FIG. 3). Through random selection from the enriched final sublibrary, finally, Fc variants WHFc1 (G236A/Q311R/P396L/M428L) and WHFc5 (A231V/G236W/F243L/Q311R/R355L/P396L/M428L) with both improved binding capacity to FcγRIIa and pH-dependent FcRn binding capacity were obtained (Table 1).

TABLE 1

| Variant | Introduced |
|---|---|
| name | mutation |
| WHFc1 | G236A/Q311R/P396L/M428L |
| WHFc5 | A231V/G236W/F243L/Q311R/R355L/P396L/M428L |

### Example 4. Expression and Purification of Trastuzumab-Fc Variants with Individual Amino Acid Substitutions

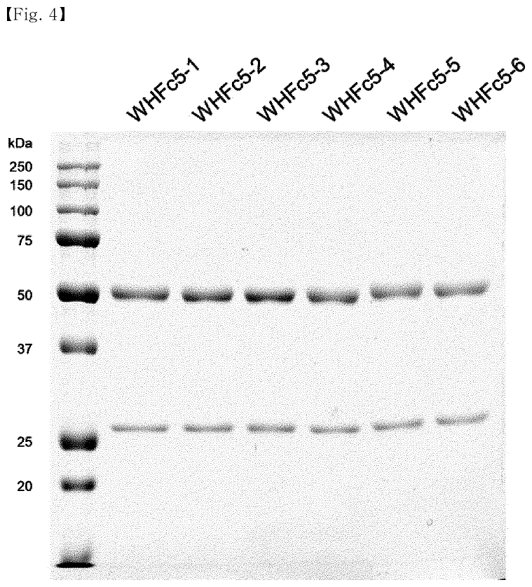

To evaluate the effect of individual amino acid substitutions of the Fc variant WHFc5 obtained in Example 3 above on the binding capacity to FcγRIIa, expression vectors pMAZ-Trastuzumab-HC-WHFc5-1, pMAZ-Trastuzumab-HC-WHFc5-2, pMAZ-Trastuzumab-HC-WHFc5-3, pMAZ-Trastuzumab-HC-WHFc5-4, pMAZ-Trastuzumab-HC-WHFc5-5, and pMAZ-Trastuzumab-HC-WHFc5-6 were constructed by substitution to Trastuzumab Fc from five variants (WHFc5-1: G236W/F243L/Q311R/R355L/P396L/M428L; WHFc5-2: A231V/F243L/Q311R/R355L/P396L/M428L; WHFc5-3: A231V/G236W/Q311R/R355L//P396L/M428L; WHFc5-4: A231V/G236W/F243L/Q311R/P396L/M428L; and WHFc5-5: A231V/G236W/F243L/Q311R/R355L/M428L) that reverted amino acid substitutions in WHFc5 to a wild type, and a variant WHFc5-6 (A231V/G236A/F243L/Q311R/R355L/P396L/M428L) substituted with G236A mutation in WHFc1 (Table 2). Heavy chain genes and light chain genes of the respective variants were first mixed in 3 ml of a Freestyle 293 expression culture medium (Gibco, 12338-018) at a 1:1 ratio, and mixed at a ratio of PEI:variant gene=4:1, left at room temperature for 20 minutes, and then transfected into Expi293F cells subcultured the previous day at a density of 2×10⁶ cells/ml. Thereafter, the transfected cells were incubated for 7 days under conditions of 37° C., 125 rpm, and 8% CO₂ in a CO₂ shaking incubator, and centrifuged at 6000×g for 15 minutes to take only the supernatant. Thereafter, 30 ml of the taken culture supernatant and 1.25 ml of 25×PBS were mixed and then filtered using a 0.2 μm bottle top filter (Corning, 430513). 100 μl of Protein A resin was added to the filtered culture medium, stirred at 4° C. for 16 hours, and then packed in a disposable polypropylene column to recover the resin. The resin was washed with 5 ml of 1×PBS (pH 7.4), eluted with 3 ml of 100 mM glycine (pH 2.7) buffer, and then neutralized using 1 M Tris-HCl (pH 8.0). The buffer was exchanged with 1×PBS (pH 7.4) using Amicon Ultra-4 centrifugal filter units 3K (Merck Millipore, UFC800324), and it was confirmed through SDS-PAGE gel that the antibody Trastuzumab-Fc variants were successfully purified with high purity (FIG. 4).

TABLE 2

| Variant | Introduced |
|---|---|
| name | mutation |
| WHFc5-1 | G236W/F243L/Q311R/R355L/P396L/M428L |
| WHFc5-2 | A231V/F243L/Q311R/R355L/P396L/M428L |
| WHFc5-3 | A231V/G236W/Q311R/R355L//P396L/M428L |
| WHFc5-4 | A231V/G236W/F243L/Q311R/P396L/M428L |
| WHFc5-5 | A231V/G236W/F243L/Q311R/R355L/M428L |
| WHFc5-6 | A231V/G236A/F243L/Q311R/R355L/P396L/M428L |

### Example 5. Evaluation of Effect of Individual Amino Acid Substitutions on FcγRIIa Binding

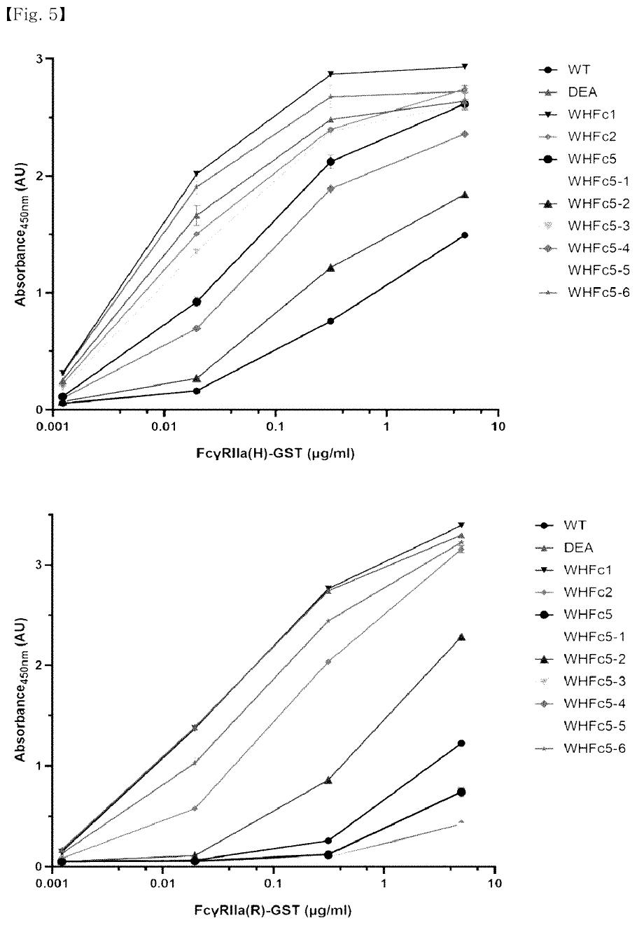

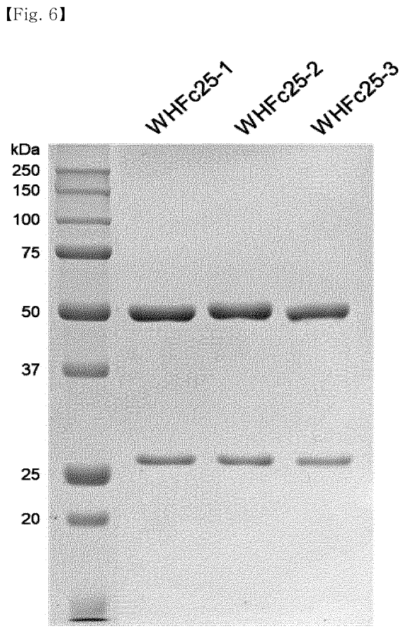

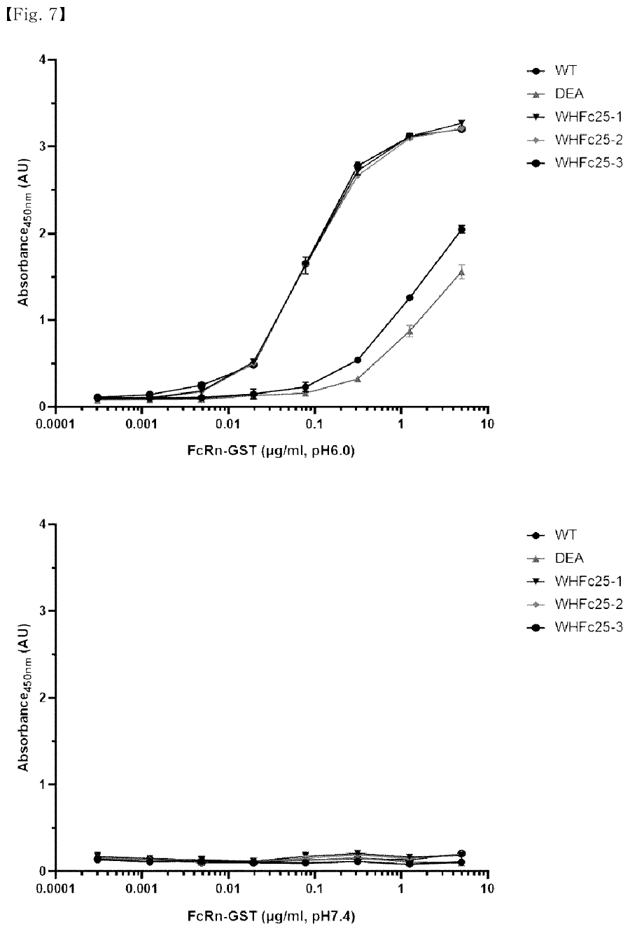

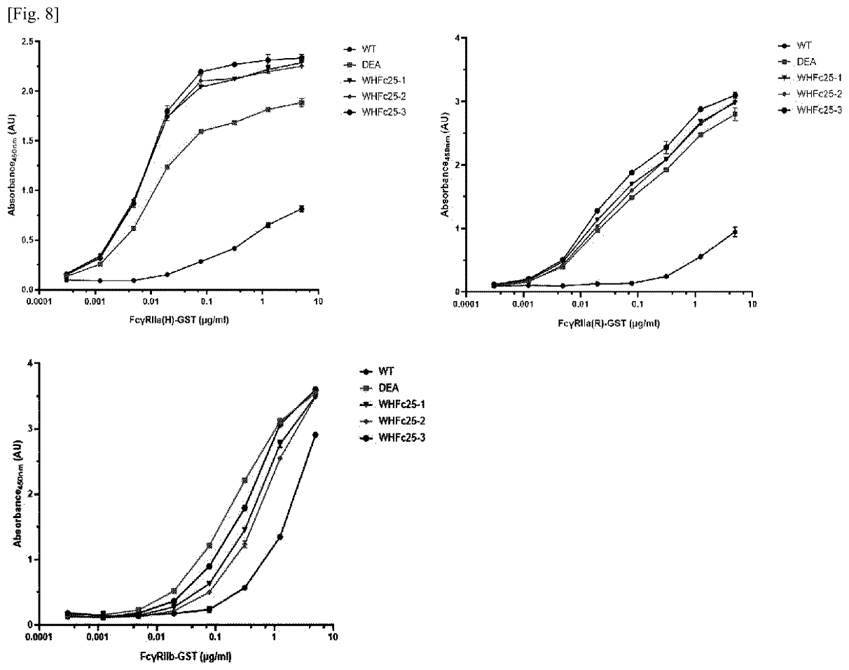

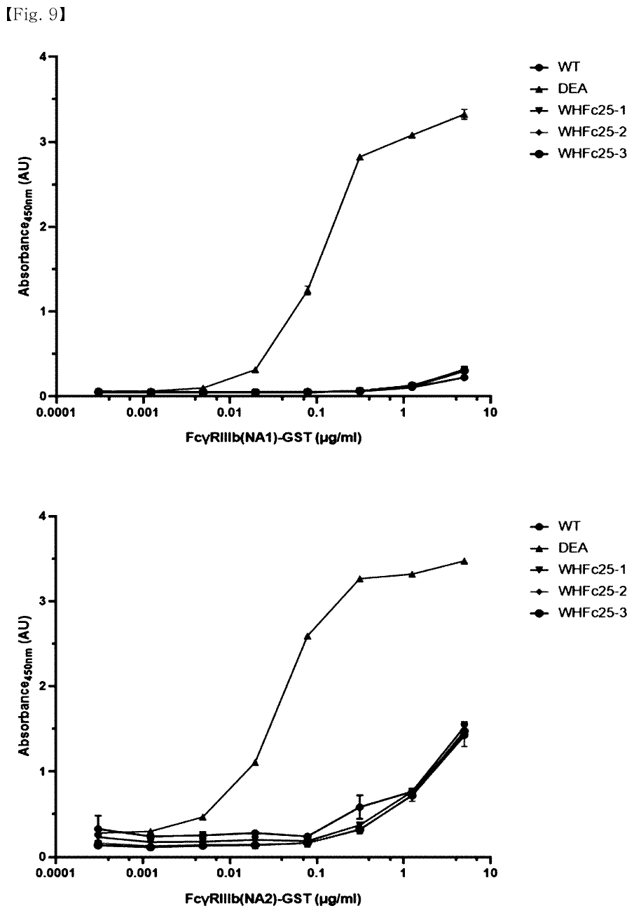

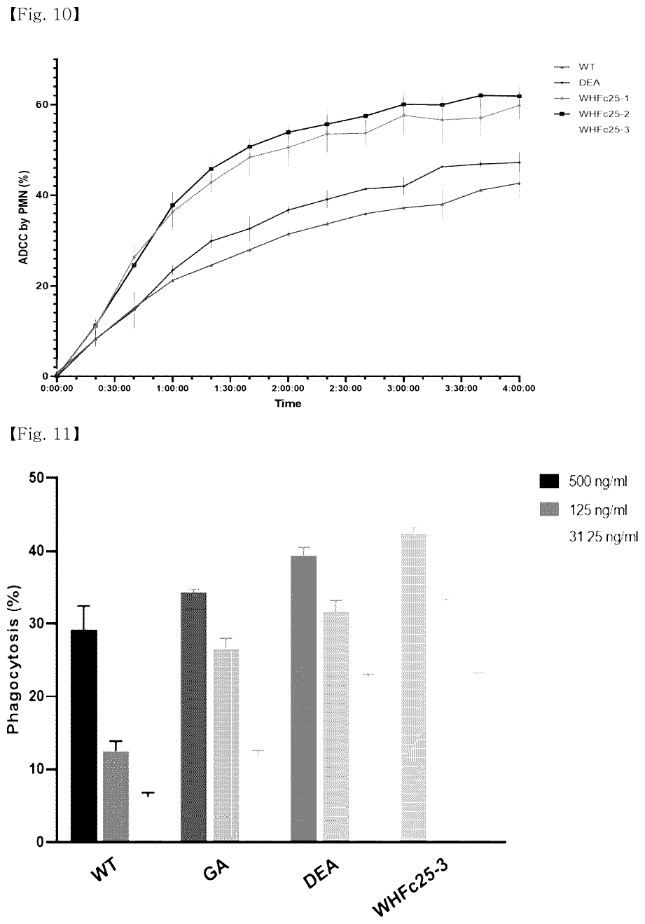

To confirm the binding capacity to FcγRIIa of the Trastuzumab-Fc variants containing the Fc variants of Table 2 prepared in Example 4 above, ELISA analysis was performed. Specifically, 50 μl each of HER2 diluted in 0.4 μg/ml of 0.05 M Na₂CO₃ (pH 9.6) was incubated and immobilized in a flat bottom polystyrene High Bind 96 well microplate (Costar, 3590) for 16 hours at 4° C., and then blocked for 2 hours at room temperature with 4% skim milk (GenomicBase, SKI400) diluted in 100 μl of 1×PBS (pH 7.4). After washed 4 times with 150 μl of 0.05% PBST (1×PBS, 0.05% Tween 20 (Sigma-Aldrich, P1379-1L)) (pH 7.4), 50 μl of Trastuzumab-Fc variants diluted to 10 μg/ml in 1% skim milk diluted in 1×PBS (pH 7.4) were dispensed into each well and reacted at room temperature for 1 hour. After washed 4 times with 100 μl of 0.05% PBST (pH 6.0/pH 7.4), 50 μl of hFcγRs-GST (hFcγRIIa-131H-GST and hFcγRIIa-131R-GST) serially diluted with 1% skim milk (pH 6.0/pH 7.4) diluted in 1×PBS were dispensed into each well and reacted at room temperature for 1 hour. After washed 4 times with 100 μl of 0.05% PBST (pH 7.4), antibody reaction was performed for 1 hour at room temperature using 50 μl of anti-GST-HRP conjugate (GE Healthcare, RPN1236V) and washed four times with 100 μl of 0.05% PBST (pH 7.4). 50 μl each of a 1-Step Ultra TMB-ELISA substrate solution (Thermo Fisher Scientific, 34028) was added to develop color, and then added with 50 μl each of 2 M H₂SO₄ to terminate the reaction. Absorbance was measured using an Epoch microplate spectrophotometer (BioTek). As a result, it was shown that among the individual amino acid substitutions, A231V, R355L, and P396L improved the binding to hFcγRIIa, whereas F243L decreased the binding to hFcγRIIa (FIG. 5), and G236A further increased the binding capacity to hFcγRIIa compared to G236W.

### Example 6. Expression and Purification of Trastuzumab-Fc Variants with Individual Amino Acid Substitution Combinations

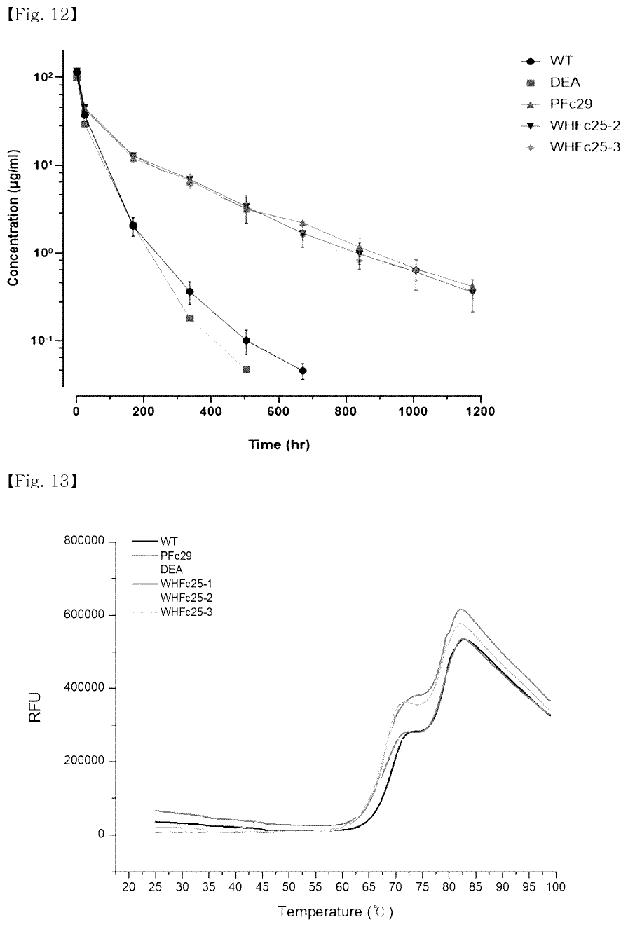

Expression vectors pMAZ-Trastuzumab-HC-WHFc25-1, pMAZ-Trastuzumab-HC-WHFc25-2, and pMAZ-Trastuzumab-HC-WHFc25-3 were constructed by substitution to Trastuzumab Fc with Fc variants combined with individual amino acid substitutions that were confirmed to have improved binding capacity to hFcγRIIa in Example 5 above, WHFc25-1 (A231V/G236A/Q311R/P396L/M428L), WHFc25-2 (G236A/Q311R/R355L/P396L/M428L), and WHFc25-3 (A231V/G236A/Q311R/R355L/P396L/M428L) (Table 3). Heavy chain genes and light chain genes of the respective variants were first mixed in 3 ml of a Freestyle 293 expression culture medium (Gibco, 12338-018) at a 1:1 ratio, and mixed at a ratio of PEI:variant gene=4:1, left at room temperature for 20 minutes, and then transfected into Expi293F cells subcultured the previous day at a density of 2×10⁶ cells/ml. Thereafter, the transfected cells were incubated for 7 days under conditions of 37° C., 125 rpm, and 8% CO₂ in a CO₂ shaking incubator, and centrifuged at 6000×g for 15 minutes to take only the supernatant. Thereafter, 30 ml of the taken culture supernatant and 1.25 ml of 25×PBS were mixed and then filtered using a 0.2 μm bottle top filter (Corning, 430513). 100 μl of Protein A resin was added to the filtered culture medium, stirred at 4° C. for 16 hours, and then packed in a disposable polypropylene column to recover the resin. The resin was washed with 5 ml of 1×PBS (pH 7.4), eluted with 3 ml of 100 mM glycine (pH 2.7) buffer, and then neutralized using 1 M Tris-HCl (pH 8.0). The buffer was exchanged with 1×PBS (pH 7.4) using Amicon Ultra-4 centrifugal filter units 3K (Merck Millipore, UFC800324), and it was confirmed through SDS-PAGE gel that the antibody Trastuzumab-Fc variants were successfully purified with high purity (FIG. 6).

TABLE 3

| Variant | Introduced |
|---|---|
| name | mutation |
| WHFc25-1 | A231V/G236A/Q311R/P396L/M428L |
| WHFc25-2 | G236A/Q311R/R355L/P396L/M428L |
| WHFc25-3 | A231V/G236A/Q311R/R355L/P396L/M428L |

### Example 7. Analysis of pH-Dependent FcRn Binding Capacity of Trastuzumab-Fc Variants with Individual Amino Acid Substitution Combinations

The pH-dependent human FcRn binding capacity of Trastuzumab variants containing Fc variants combined with individual amino acid substitution mutations (Table 3) in Example 6 above was analyzed using ELISA. Specifically, 50 μl each of HER2 diluted in 4 μg/ml of 0.05 M Na₂CO₃ (pH 9.6) was incubated and immobilized in a flat bottom polystyrene High Bind 96 well microplate (Costar, 3590) for 16 hours at 4° C., and then blocked for 2 hours at room temperature with 4% skim milk (GenomicBase, SKI400) diluted in 100 μl of 1×PBS (pH 7.4). After washed 4 times with 150 μl of 0.05% PBST (pH 7.4), 50 μl of Trastuzumab Fc variants diluted to 10 μg/ml in 1% skim milk diluted with 1×PBS (pH 7.4) were dispensed into each well and reacted at room temperature for 1 hour. After washed 4 times with 100 μl of 0.05% PBST (pH 6.0/pH 7.4), 50 μl of hFcRn-GST serially diluted with 1% skim milk (pH 6.0/pH 7.4) diluted in 1×PBS were dispensed into each well and reacted at room temperature for 1 hour. After washed four times with 100 μl of 0.05% PBST (pH 7.4), the antibody reaction was performed for 1 hour at room temperature using 50 μl of anti-GST-HRP conjugate (GE Healthcare, RPN1236V) diluted in 1% skim milk (pH 6.0/pH 7.4) diluted in 1×PBS and washed four times with 100 μl of 0.05% PBST (pH 6.0/pH 7.4). 50 μl each of a 1-Step Ultra TMB-ELISA substrate solution (Thermo Fisher Scientific, 34028) was added to develop color, and then added with 50 μl each of 2 M H₂SO₄ to terminate the reaction. Absorbance was measured using an Epoch microplate spectrophotometer (BioTek).

As a result, the Trastuzumab-Fc variants introduced with WHFc25-1, WHFc25-2 and WHFc25-3 variants of the present disclosure showed a significantly improved binding capacity to hFcRn at pH 6.0 compared to pH 7.4, compared to a Trastuzumab-Fc variant introduced i with a conventional DEA variant (S239D/1332E/G236A), showed an increased pH-dependent binding capacity, and showed a significantly improved pH-dependent FcRn binding capacity compared to wild-type Trastuzumab (FIG. 7).

### Example 8. Analysis of FcγRs Binding Capacity of Trastuzumab-Fc Variants with Individual Amino Acid Substitution Combinations

### 8-1. Analysis of Binding Capacity to FcγRIIa

To determine the binding capacity to FcγRIIa of Trastuzumab-Fc containing Fc variants combined with individual amino acid substitution mutations, ELISA analysis was performed. Specifically, 50 μl each of HER2 diluted in 0.4 μg/ml of 0.05 M Na₂CO₃ (pH9.6) was incubated and immobilized in a flat bottom polystyrene High Bind 96 well microplate (Costar, 3590) for 16 hours at 4° C., and then blocked for 2 hours at room temperature with 4% skim milk (GenomicBase, SKI400) diluted in 100 μl of 1×PBS (pH 7.4). After washed 4 times with 150 μl of 0.05% PBST (1×PBS and 0.05% Tween 20 (Sigma-Aldrich, P1379-1L)) (pH 7.4), 50 μl of Trastuzumab-Fc variants diluted to 10 μg/ml in 1% skim milk diluted with 1×PBS (pH 7.4) were dispensed into each well and reacted at room temperature for 1 hour. After washed 4 times with 100 μl of 0.05% PBST (pH 6.0/pH 7.4), 50 μl of hFcγRs-GST (hFcγRIIa-131H-GST, hFcγRIIa-131R-GST and hFcγRIIb-GST) serially diluted in 1% skim milk (pH 6.0/pH 7.4) diluted in 1×PBS were dispensed into each well and reacted at room temperature for 1 hour. After washed 4 times with 100 μl of 0.05% PBST (pH 7.4), antibody reaction was performed for 1 hour at room temperature using 50 μl of anti-GST-HRP conjugate (GE Healthcare, RPN1236V) and washed four times with 100 μl of 0.05% PBST (pH 7.4). 50 μl each of a 1-Step Ultra TMB-ELISA substrate solution (Thermo Fisher Scientific, 34028) was added to develop color, and then added with 50 μl each of 2 M H₂SO₄ to terminate the reaction. Absorbance was measured using an Epoch microplate spectrophotometer (BioTek).

As a result, it was shown that the Trastuzumab-Fc variants of the present disclosure had a significantly improved binding capacity to hFcγRIIa-131H and hFcγRIIa-131R compared to the wild-type Trastuzumab-Fc variant, and it was shown that the Trastuzumab-Fc variants of the present disclosure had a significantly increased binding capacity compared to the binding capacity to hFcγRIIa-131H and hFcγRIIa-131R of Trastuzumab-Fc variants into which the conventional DEA (DE: S239D/1332E/G236A) variant was introduced (FIG. 8). In addition, it was confirmed that the Trastuzumab-Fc variants of the present disclosure exhibited a lower hFcγRIIb binding capacity compared to the Trastuzumab-Fc variant into which the DEA variant was introduced (FIG. 8).

### 8-2. Analysis of Binding Capacity to FcγRIIIb

To determine the binding capacity to FcγRIIIb of Trastuzumab-Fc containing Fc variants combined with individual amino acid substitution mutations, ELISA analysis was performed. Specifically, 50 μl each of HER2 diluted in 4 g/ml of 0.05 M Na₂CO₃ (pH 9.6) was incubated and immobilized in a flat bottom polystyrene High Bind 96 well microplate (Costar, 3590) for 16 hours at 4° C., and then blocked for 2 hours at room temperature with 4% skim milk (GenomicBase, SKI400) diluted in 100 μl of 1×PBS (pH 7.4). After washed 4 times with 150 μl of 0.05% PBST (1×PBS and 0.05% Tween 20 (Sigma-Aldrich, P1379-1L)) (pH 7.4), 50 μl of Trastuzumab-Fc variants diluted to 10 μg/ml in 1% skim milk diluted with 1×PBS (pH 7.4) were dispensed into each well and reacted at room temperature for 1 hour. After washed 4 times with 100 μl of 0.05% PBST (pH 6.0/pH 7.4), 50 μl of hFcγRs-GST (hFcγRIIIb-NA1-GST and hFcγRIIIb-NA2-GST) serially diluted in 1% skim milk (pH 6.0/pH 7.4) diluted in 1×PBS were dispensed into each well and reacted at room temperature for 1 hour. After washed 4 times with 100 μl of 0.05% PBST (pH 7.4), antibody reaction was performed for 1 hour at room temperature using 50 μl of anti-GST-HRP conjugate (GE Healthcare, RPN1236V) and washed four times with 100 μl of 0.05% PBST (pH 7.4). 50 μl each of a 1-Step Ultra TMB-ELISA substrate solution (Thermo Fisher Scientific, 34028) was added to develop color, and then added with 50 μl each of 2 M H₂SO₄ to terminate the reaction. Absorbance was measured using an Epoch microplate spectrophotometer (BioTek).

As a result, it was confirmed that the Trastuzumab-Fc variants of the present disclosure had a reduced or similar binding capacity to hFcγRIIIb-NA1 and hFcγRIIIb-NA2 compared to the wild-type Trastuzumab-Fc variant, whereas it was shown that the binding capacity to hFcγRIIIb-NA1 and hFcγRIIIb-NA2 of Trastuzumab-Fc variants into which the conventional DEA variant was introduced was significantly increased compared to the wild-type Trastuzumab-Fc variant (FIG. 9).

### Example 9. Analysis of ADCC Effect on Neutrophils of Trastuzumab-Fc Variants with Individual Amino Acid Substitution Combinations

An experiment was conducted to compare antibody-dependent cell-mediated cytotoxicity (ADCC) effects of neutrophils (polymorphonuclear cells, PMN) against cancer cells by Trastuzumab-Fc variants with individual amino acid substitution combinations. First, to isolate neutrophils from a blood sample, 3 ml of Histopaque-1119 (Sigma-Aldrich, 1119-100ML), 3 ml of Histopaque-1077 (Sigma-Aldrich, 1077-100ML), and 4 ml of blood were sequentially stacked in a 15-ml conical tube, and then centrifuged at 700×g for 30 minutes, and only a granulocyte layer was taken. The neutrophils were harvested after lysing red blood cells with a RBC lysis buffer (eBioscience, 00-4333-57) and incubated in a culture medium containing 50 ng/ml IFN-γ (BioLegend, 570202) and 10 ng/ml G-CSF (PeproTech, 300-23-2UG) at 37° C. and 5% CO₂ for 16 hours. Meanwhile, SK-BR-3 cells incubated at 1×10⁴ cells/well in a 96 Well Black/Clear Bottom Plate (Thermo Scientific, 165305) at the previous day were stained with 2 μM calcein-AM (InivivoGen, C3100MP) for 30 minutes. The incubated neutrophils were incubated with SK-BR-3 stained at 2×10⁵ cells/well, and added with Trastuzumab-Fc variants diluted to 5 μg/ml, respectively, and live-cell imaging was performed using Lionheart FX (BioTek). In addition, cell death of SK-BR-3 was analyzed and graphed using fluorescence imaging.

As a result, it was confirmed that the obtained Trastuzumab-Fc variants of the present disclosure showed significantly improved neutrophil ADCC efficiency compared to the wild-type Trastuzumab-Fc variant, and had improved neutrophil ADCC efficiency compared to a Trastuzumab-Fc variant introduced with a conventional DEA variant (FIG. 10).

### Example 10. Analysis of Macrophage ADCP Efficiency of Trastuzumab-Fc Variants with Individual Amino Acid Substitution Combinations

To evaluate the ADCP efficiency of macrophages by Trastuzumab-Fc variants with individual amino acid substitution combinations, to obtain monocytes from a blood sample, 6 ml of Histopque-1077 (Sigma-Aldrich, 1077-100ML) and 8 ml of blood diluted 1:1 with PBS were sequentially stacked in a 15 ml conical tube, and centrifuged at 1000×g for 10 minutes, and only PBMCs were taken. To remove platelets, the PBMCs were dissolved in 50 ml of PBS, centrifuged at 100×g for 10 minutes, and the supernatant was removed. Monocytes were isolated from PBMCs using CD14 MicroBeads (Miltenyi Biotec, 130-050-201) and differentiated into macrophages for one week with 50 ng/ml GM-CSF (PeproTech, 300-03). Meanwhile, SK-BR-3 was stained on the cell surface with 2 μM PKH67 (Sigma-Aldrich, MIDI67-1KT), mixed with the differentiated macrophages at a ratio of 1:5, and then incubated with the Trastuzumab-Fc variant (WHFc25-3) of the present disclosure at 37° C. and 5% CO₂ for 4 hours. Thereafter, macrophages were stained with anti-CD11b-APC antibody (BioLegend, 301309) and anti-CD14-APC antibody (BioLegend, 301807) and analyzed by FACS, and the obtained data were analyzed using FlowJo software.

As a result, it was confirmed that the Trastuzumab-Fc variant introduced with the WHFc25-3 variant of the present disclosure exhibited significantly improved ADCP efficiency compared to the wild type, and exhibited similar ADCP efficiency to the Trastuzumab-Fc variant introduced with the conventional DEA variant (FIG. 11).

### Example 11. Evaluation of Half-Life in Blood of Trastuzumab-Fc Variants with Individual Amino Acid Substitution Combinations

To evaluate the in vivo half-life in blood of Trastuzumab-Fc variants with individual amino acid substitution combinations, pharmacokinetic profile analysis was performed using a hFcRn transgenic mouse. Specifically, Trastuzumab-Fc variants expressed in animal cells and purified by Protein A affinity chromatography were further purified by size-exclusion chromatography (SEC) using the NGC Chromatography System (Bio Rad). Each Trastuzumab-Fc variant was intravenously administered to a Tg276 mouse at a dose of 5 mg/kg, and then about 100 μl of the blood was collected at 30 minutes and 24 hours and the serum was taken. Thereafter, to obtain the serum by collecting the blood at intervals of 7 days and analyze the content of residual Trastuzumab-Fc variants in the body by quantitative ELISA, 50 μl each of HER2 diluted in 0.4 μg/ml of 0.05 M Na₂CO₃ (pH 9.6) was incubated and immobilized in a flat bottom polystyrene High Bind 96 well microplate (Costar, 3590) for 16 hours at 4° C., and then blocked for 2 hours at room temperature with 4% skim milk (GenomicBase, SKI400) diluted in 100 μl of 1×PBS (pH 7.4). After washed 4 times with 150 μl of 0.05% PBST (1×PBS, and 0.05% Tween 20 (Sigma-Aldrich, P1379-1L)) (pH 7.4), 50 μl of the serum serially diluted in 1% skim milk diluted with 1×PBS (pH 7.4) was dispensed and reacted at room temperature for 1 hour. At this time, the serum reacted with the Trastuzumab-Fc variant serially diluted from the concentration of 1 μg/ml for quantification and used to obtain a standard curve. After washed four times with 100 μl of 0.05% PBST (pH 6.0/pH 7.4), the reaction was performed for 1 hour at room temperature with 50 μl of Peroxidase AffiniPure F(ab′)₂ Fragment Goat Anti-Human IgG (H+L) (Jackson ImmunoResearch Laboratories, 109-036-003), and then washed four times with 100 μl of 0.05% PBST (pH 7.4). 50 μl each of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek). Using the obtained results, the concentration of the residual Trastuzumab-Fc variants in each sample was calculated and graphed to measure the half-life in blood.

TABLE 4

| | T_{1/2}(h) |
|---|---|
| Trastuzumab | 57.4 ± 2.9 |
| Trastuzumab-DEA (Xencor) | 51.5 ± 0.5 |
| Trastuzumab-PFc29 | 166.7 ± 11.3 |
| Trastuzumab-WHFc25-2 | 174.9 ± 19.4 |
| Trastuzumab-WHFc25-3 | 150.8 ± 4.0 |

As a result, it was shown that the half-life in blood of the Trastuzumab-Fc variants introduced with the variants of the present disclosure was significantly longer than that of the wild-type Trastuzumab, and particularly, it was shown that the half-life in blood of the Trastuzumab-Fc variants was significantly improved even compared to the Trastuzumab-Fc variant introduced with the DEA variant reported in the previous study (FIG. 12 and Table 4).

### Example 12. Evaluation of Thermal Stability of Trastuzumab-Fc Variants with Individual Amino Acid Substitution Combinations

To evaluate the thermal stability of Trastuzumab-Fc containing Fc variants combined with individual amino acid substitution mutations, thermofluor analysis was performed. Specifically, 45 μl of protein (Trastuzumab-Fc variant) diluted in PBS to 5 μM and 5 μl of SYPRO Orange (Invitrogen, S6651) diluted 200× in PBS were placed in a white PCR plate (Thermo Scientific, AB0900W) and sealed with an optically clear sealing film (Thermo Scientific, AB1170). The PCR plate was heated from 25° C. to 99.9° C. at a ramp rate of 0.03° C./s and fluorescence was analyzed using a QuantStudio 3 Real-Time PCR System (Applied Biosystems, A28567). The fluorescence signal was background subtracted using a PBS sample, and graphed using the temperature as a variable, and a midpoint of a sigmoidal transition curve of the graph, which was a melting temperature (Tm) of each protein, was determined using OriginPro software. The analysis was performed three times.

TABLE 5

| | Tₘ(° C.) |
|---|---|
| Trastuzumab | 69.5 ± 0.053 |
| Trastuzumab-PFc29 | 68.5 ± 0.296 |
| Trastuzumab-DEA | 50.9 ± 0.099 |
| Trastuzumab-WHFc25-1 | 68.2 ± 0.009 |
| Trastuzumab-WHFc25-2 | 69.1 ± 0.081 |
| Trastuzumab-WHFc25-3 | 68.4 ± 0.149 |

As a result, it was shown that the Trastuzumab variants of the present disclosure were not decreased in thermal stability compared to the wild-type Trastuzumab variant, whereas the Trastuzumab-Fc variant introduced with the conventional DEA variant was significantly decreased in thermal stability compared to the wild-type (FIG. 13 and Table 5).

1. A human antibody Fc domain variant in which amino acids at positions 231 and 355 numbered according to the Kabat numbering system in a wide-type human antibody Fc domain are substituted with sequences different from wild-type amino acids.

2. The human antibody Fc domain variant of claim 1, wherein one or more amino acids selected from the group consisting of amino acids at positions 231, 236, 311, 355, 396 and 428 numbered according to the Kabat numbering system in the wide-type human antibody Fc domain are substituted with sequences different from the wild-type amino acids.

3. The human antibody Fc domain variant of claim 1, wherein the human antibody Fc domain variant includes one or more amino acid substitutions selected from the group consisting of A231V, G236A, Q311R, R355L, P396L and M428L.

4. The human antibody Fc domain variant of claim 1, wherein the human antibody Fc domain variant includes amino acid substitutions of A231V, G236A, Q311R, P396L and M428L.

5. The human antibody Fc domain variant of claim 1, wherein the human antibody Fc domain variant includes amino acid substitutions of G236A, Q311R, R355L, P396L and M428L.

6. The human antibody Fc domain variant of claim 1, wherein the human antibody Fc domain variant includes amino acid substitutions of A231V, G236A, Q311R, R355L, P396L and M428L.

7. The human antibody Fc domain variant of claim 1, wherein the human antibody Fc domain variant has an improved binding capacity to FcγRIIa compared to a wild-type human antibody Fc domain.

8. The human antibody Fc domain variant of claim 1, wherein the human antibody Fc domain variant has an increased A/I ratio compared to the wild-type human antibody Fc domain.

9. The human antibody Fc domain variant of claim 1, wherein the human antibody Fc domain variant has an improved selective binding capacity to FcγRIIa relative to FcγRIIIb compared to the wild-type human antibody Fc domain.

10. The human antibody Fc domain variant of claim 1, wherein the human antibody Fc domain variant has an improved effector function compared to the wild-type human antibody Fc domain.

11. The human antibody Fc domain variant of claim 10, wherein the effector function is an Fc-mediated effector function selected from, antibody-dependent cellular cytotoxicity (ADCC), antibody-dependent cell-mediated phagocytosis (ADCP), C1q-binding, complement activation, complement dependent cytotoxicity (CDC), Fc-receptor binding including Fc-gamma receptor binding, protein A-binding, protein G-binding, complement dependent cell-mediated cytotoxicity (CDCC), complement-enhanced cytotoxicity, opsonization, Fc-containing polypeptide internalization, target downmodulation, ADC uptake, induction of apoptosis, cell death, cell cycle arrest, and any combination thereof.

12. The human antibody Fc domain variant of claim 1, wherein the human antibody Fc domain variant exhibits low binding affinity to FcRn at pH 7.0 to 7.8 compared to the wild-type human antibody Fc domain.

13. The human antibody Fc domain variant of claim 1, wherein the human antibody Fc domain variant exhibits high binding affinity to FcRn at pH 5.6 to 6.5 compared to the wild-type human antibody Fc domain.

14. The human antibody Fc domain variant of claim 1, wherein the human antibody Fc domain variant has an increased in vivo half-life compared to the wild-type human antibody Fc domain.

15. An antibody or immunologically active fragment thereof with an improved binding capacity to an Fc gamma receptor comprising the Fc domain variant of claim 1.

16. The antibody or immunologically active fragment thereof of claim 15, wherein the antibody or immunologically active fragment thereof has an increased in vivo half-life compared to a wild-type human antibody.

17. A bioactive polypeptide conjugate having an increased in vivo half-life by binding the human antibody Fc domain variant of claim 1 and a bioactive polypeptide.

18. The bioactive polypeptide conjugate of claim 17, wherein the bioactive polypeptide is selected from the group consisting of human growth hormone, growth hormone-releasing hormone, growth hormone-releasing peptide, interferon, colony-stimulating factor, interleukin, interleukin soluble receptor, TNF soluble receptor, glucocerebrosidase, macrophage activator, macrophage peptide, B-cell factor, T-cell factor, protein A, allergy suppressor, necrosis glycoprotein, immunotoxin, lymphotoxin, tumor necrosis factor, tumor suppressor, metastasis growth factor, alpha-1 antitrypsin, albumin, apolipoprotein-E, erythropoietin, hyperglycosylated erythropoietin, blood factor VII, blood factor VIII, blood factor IX, plasminogen activator, urokinase, streptokinase, protein C, C-reactive protein, renin inhibitor, collagenase inhibitor, superoxide dismutase, leptin, platelet-derived growth factor, epidermal growth factor, osteogenic growth factor, bone formation-promoting protein, calcitonin, insulin, insulin derivatives, glucagon, glucagon like peptide-1, atriopeptin, cartilage-inducing factor, connective tissue activator, follicle-stimulating hormone, luteinizing hormone, follicle-stimulating hormone-releasing hormone, nerve growth factor, parathyroid hormone, relaxin, secretin, somatomedin, insulin-like growth factor, corticosteroid, cholecystokinin, pancreatic polypeptide, gastrin-releasing peptide, corticotropin-releasing factor, thyroid-stimulating hormone, receptors, receptor antagonist, cell surface antigen, monoclonal antibody, polyclonal antibody, antibody fragments, and virus-derived vaccine antigens.

19. A nucleic acid molecule encoding the human antibody Fc domain variant of claim 1, or an antibody or immunologically active fragment thereof comprising the human antibody Fc domain variant.

20. A pharmaceutical composition for preventing or treating cancer comprising the human antibody Fc domain variant of claim 1, an antibody or immunologically active fragment thereof comprising the human antibody Fc domain variant, or a bioactive polypeptide conjugate having an increased in vivo half-life by binding the human antibody Fc domain variant and a bioactive polypeptide as an active ingredient.

21. A method for preparing a human antibody Fc domain variant comprising:
a) incubating a host cell including a vector including a nucleic acid molecule encoding the human antibody Fc domain variant of claim 1; and
b) recovering a polypeptide expressed by the host cell.

22. A method for preparing an antibody specific for an Fc gamma receptor, comprising:
a) incubating a host cell including a vector including a nucleic acid molecule encoding the antibody or immunologically active fragment thereof of claim 15; and
b) purifying the antibody expressed by the host cell.

23. (canceled)

24. (canceled)

25. A method for treating cancer comprising administering the human antibody Fc domain variant of claim 1, an antibody or immunologically active fragment thereof comprising the human antibody Fc domain variant, or a bioactive polypeptide conjugate having an increased in vivo half-life by binding the human antibody Fc domain variant and a bioactive polypeptide in a pharmaceutically effective amount, to a subject with cancer.
